(19) United States
(12) Patent Application Publication
(10) Pub. No.: US 2026/0118544 A1
(43) Pub. Date: Apr. 30, 2026

(54) SYSTEMS AND METHODS FOR EVALUATING HYDROGEN GENERATION POTENTIAL USING MAGNETIC SUSCEPTIBILITY AND GRAVITY MEASUREMENTS FOR NATURAL HYDROGEN EXPLORATION

(71) Applicant: Koloma, Inc., Denver, CO (US)

(72) Inventors: Thomas Darrah, Westerville, OH (US); Brent Lary, Columbus, OH (US); Christopher Gardner, Columbus, OH (US); Colin Whyte, Grove City, OH (US); William Eymold, Columbus, OH (US); Peter Kras, Columbus, OH (US)

(21) Appl. No.: 18/943,774

(22) Filed: Nov. 11, 2024

Related U.S. Application Data
(60) Provisional application No. 63/713,044, filed on Oct. 28, 2024.

(51) Int. Cl. G01V 3/38; G01N 27/72; G01N 33/24; G01V 3/18

(52) U.S. Cl. CPC G01V 3/38; G01N 27/72; G01N 33/24; G01V 3/18

(57) ABSTRACT

A method for evaluating geological hydrogen source rock in a region is provided. The method includes: collecting magnetic susceptibility data from a plurality of subsurface locations in the region; receiving geophysical survey data of the region, wherein the geophysical survey data comprises geophysical data collected from above ground; tying the magnetic susceptibility data of the plurality of subsurface locations to the geophysical survey data; generating a geologic map of igneous rock, metamorphic rock, or mafic mineral-or iron-rich sedimentary rock within the region based on the magnetic susceptibility data using a model; and determining, based on the geologic map, a target zone of the geological hydrogen source rock with a potential for hydrogen production or with the potential for hydrogen accumulation.

### CROSS-REFERENCE TO RELATED APPLICATIONS

This application claims benefit to U.S. Provisional Application No. 63/713,044, filed on Oct. 28, 2024, the entire disclosure of which is incorporated herein by reference.

### FIELD OF THE INVENTION

Embodiments of the present disclosure relate generally to the field of energy extraction, geology, geochemistry, mineralogy, or geologic hydrogen extraction. Some embodiments disclose methods of evaluating rock material for elemental, chemical (e.g., oxide), or mineralogical composition in order to evaluate the geologic timing of hydrogen generation from a given rock sample for the purposes outlined above and below. More particularly, the present disclosure relates to methods for identifying, evaluating, and high-grading rocks associated with one or more past generations of hydrogen from geologic materials to improve exploration for geologic hydrogen, or combinations thereof.

This section is intended to introduce various aspects of the technical field, which may be associated with embodiments described in this disclosure. Thus, the forgoing discussion in this section provides a framework for better understanding the disclosure, and is not to be viewed as an admission of prior art.

### BACKGROUND

Hydrogen is a carbon-free energy carrier and chemical feedstock that holds promise as an energy source. Hydrogen can be generated using sustainable energy sources such as geothermal, solar, wind, and hydroelectric power. The disclosure herein relates to hydrogen produced from or generated, both naturally or through the introduction of various fluids, within the subsurface by drilling, boring, mining, or various other means of penetrating the earth.

In the production of natural resources from formations within the earth, a well or borehole is drilled into the earth to the location where the natural resource is located. These natural resources may be hydrogen, helium, carbon dioxide, nitrogen, dihydrogen sulfide, methane, or other hydrocarbon gases; or a dihydrogen sulfide reservoir, a hydrogen reservoir, a helium reservoir, a carbon dioxide reservoir, a natural gas reservoir, a reservoir rich in dihydrogen sulfide, a reservoir rich in hydrocarbons, a reservoir rich in helium. The natural resource may be fresh water, brackish water, or brine. It may be a heat source for geothermal energy, or it may be some other natural resource, ore deposit, mineral, metal, or gem that is located within the ground.

These resource-containing formations may be a few hundred feet, a few thousand feet, or tens of thousands of feet below the surface of the earth, including under the floor of a body of water (e.g., below the sea floor) or beneath other natural resources (e.g., below aquifers, lakes, mines). In addition to being at various depths within the earth, these formations may include areas of differing sizes, shapes, and volumes.

Typically, and by way of general illustration, in drilling a well, an initial borehole is formed in the earth, such as in the surface of land or seabed, and then subsequent and smaller diameter boreholes are drilled to extend the overall depth of the borehole. As the overall borehole gets deeper, its diameter becomes smaller, resulting in a telescoping assembly of boreholes with the largest diameter hole being at the top of the borehole closest to the surface of the earth.

The starting phases of a subsea drill process may be explained in general as follows. Once the drilling rig is positioned on the surface of the water over the area where drilling is to take place, an initial borehole is made by drilling a 36″ hole in the earth to a depth of about 200 to 300 ft. below the seafloor. A 30″ casing, also known as a conductor, is inserted into this initial borehole. The 30″ conductor may or may not be cemented into place. During this drilling operation, a riser is generally not used and the cuttings from the borehole (e.g., the earth and other material removed from the borehole by the drilling activity) are returned to the seafloor. Next, a 26″ diameter borehole is drilled within the 30″ casing, extending the depth of the borehole to about 1,000 to 1,500 ft. This drilling operation may also be conducted without using a riser. A 20″ casing is then inserted into the 30″ conductor and 26″ borehole and cemented into place. The 20″ casing has a wellhead secured to it. In other operations, an additional smaller diameter borehole may be drilled, and a smaller diameter casing inserted into that borehole with the wellhead being secured to that smaller diameter casing. A blow out preventer (BOP) is then secured to a riser and lowered by the riser to the sea floor, where the BOP is secured to the wellhead. From this point forward, all drilling activity in the borehole takes place through the riser and the BOP.

It should be noted that subsea drilling operations that do not employ a riser are also contemplated.

For a land-based drill process, the steps are similar, although the 30″ to 20″ large diameter tubulars are typically not used. Generally, there is a surface casing that is typically about 13⅜″ diameter that may extend from the surface such that the wellhead and BOP are extended to depths of tens of feet to hundreds of feet. One purpose of the surface casing is to meet environmental requirements to protect ground water by preventing surface casing ventflow to groundwater aquifers or to prevent surface casing ventflow of greenhouse gases or flammable gases to groundwater aquifers or the atmosphere. The surface casing should have sufficiently large diameter to allow the drill string, production equipment (e.g., electronic submersible pumps (ESPs)), and circulation mud to pass through. Below the casing, one or more different diameter intermediate casings may be used. It is understood that sections of a borehole may not be cased and are referred to as open hole. These sections can have diameters in the range of about 9″ to about 7″, although larger and smaller sizes may be used, and can extend to depths of thousands and tens of thousands of feet.

The portion of the well located within the section of the formation containing the natural resources may be referred to as the “pay zone.” Production tubing is placed inside the casing and extends from the pay zone or production zone of the borehole up to and through the wellhead on the surface. There may be a single production tubing or multiple production tubings in a single borehole, with each of the production tubing endings being at different depths.

Fluid communication between the formation and the well or borehole can be greatly increased by the use of perforations, hydraulic fracturing, or other stimulation techniques. In general, hydraulic fracturing treatments involve forcing fluids down the well or borehole and into the formation, where the fluids enter the formation and crack, e.g., by forcing the layers of rock to break apart or fracture. These fractures create channels or flow paths that may have cross sections of a few microns to a few millimeters to several millimeters in size, and potentially larger. The fractures may also extend out from the well in all directions for a few feet, several feet, and tens of feet or further. The fractures may be kept open by using a proppant (e.g., various sized sand or other mineral grains) that are forced down the well with the fracturing fluid in a single operation. The longitudinal axis of the well or borehole in the reservoir may not be vertical: it may be on an angle (either sloping up or down) or it may be horizontal.

During the drilling of wells or boreholes, drilling fluids such as water-based mud, oil-based mud, water, foam, aerated mud, air, synthetic fluids, or other fluids, herein referred to as “drilling fluid,” are often pumped down the borehole through the drill string and out into the borehole at the drill bit, then back up to the surface between the exterior of the drill string and the borehole wall. In some drilling operations, air or aerated fluid is injected through the drill string in a similar manner and can return formation fluids, including gases, to the surface. Drilling fluid may be used to lubricate the borehole, drill bit, and drill string and prevent thermal degradation of the drill bit, as well as provide a medium through which to eject drilled rock or cuttings, sediment material, or formation fluids (e.g., gases) up the borehole to the surface.

In some examples, the subsurface rock formation from which gases are extracted can include at least one of sedimentary rocks (e.g., sandstone, limestone, shales, graywacke, evaporites), metamorphic rocks (e.g., serpentinites, marbles), igneous rocks (e.g., dunite, pyroxenite, basalt, gabbros, granites, or others), or from formations containing overly thermally mature hydrocarbon fluids, hydrocarbon source rocks, coal, or graphite. Other examples can include iron-rich rock, mafic or ultramafic igneous rock, metamorphosed or hydrothermally altered mafic or ultramafic igneous rock, olivine-or pyroxene-bearing igneous, metamorphic, or sedimentary rock or sediment, metamorphosed or hydrothermally altered olivine- or pyroxene-bearing igneous, metamorphic, or sedimentary rock or sediment, serpentine mineral-bearing rock or sediment, partially or completely serpentinized rock, serpentinite, amphibole-rich igneous or metamorphic rock, amphibolite, pyrite-bearing rock, iron-rich or other metalliferous ore deposit, iron-rich sandstone, other iron-rich sedimentary rock, or iron-rich sediments.

In some examples, the source of hydrogen can include any of the sources described above (e.g., mafic or ultramafic rock) that is drilled, drilled and stimulated (e.g., hydraulic fracturing or perforation), drilled and stimulated (e.g., hydraulic fracturing or perforation) with the accompanying introduction of heat, chemicals, or fluids (e.g., carbon dioxide and/or dihydrogen sulfide), or fluids encountered while interacting with various subsurface reservoirs or geothermal systems, mining operations, water well drilling, formation waters, or any fluids exsolved from processes related to their exploration, characterization, or extraction.

### BRIEF SUMMARY

There exists a need for systems and methods for evaluating geological hydrogen source rock potential and hydrogen reservoirs within geological hydrogen systems using magnetic susceptibility and gravity (density) measurements for the purposes of identifying suitable conventional and unconventional targets for natural hydrogen exploration.

The present disclosure relates to methods for identifying, evaluating, and high-grading geological hydrogen source rocks associated with the generation of geologic hydrogen using high-resolution measurements of magnetic susceptibility, with some applications integrating gravity or mineral composition measurements. These measurements can be obtained through wireline tools, or direct analyses of drilled material, such as cuttings, whole cores, or rotary sidewall cores, or rocks or sediments collected at the surface. Another embodiment of this disclosure relates to using these measurements to calibrate, integrate, and improve the development of geophysical models and subsurface interpretations generated through the use of seismic (seismic reflection, refraction, ambient noise tomography, or tomography), ground-based-, sea-based-or airborne-magnetic, gravity, and/or gravity gradiometry surveys, or combinations thereof for the purposes of exploration for geologic hydrogen, construction of geologic hydrogen exploration models, or the identification of targets for carbon sequestration by subsurface carbon mineralization or sulfur sequestration by mineralization.

The present disclosure relates to systems and methods for identifying, evaluating, and high-grading source rocks associated with the generation of geologic hydrogen using high-resolution measurements of magnetic susceptibility and gravity. Background materials used for the basis of measurements obtained through wireline tools or direct analyses of drilled material, such as cuttings, whole core, or rotary sidewall core, or rocks or sediments collected at the surface are described herein. Similarly, the presently disclosed systems and methods use these geophysical measurements to calibrate, integrate, and improve the development of geophysical models and subsurface interpretations generated through the use of seismic (e.g., reflection, refraction, tomography), airborne magnetic, gravity, and/or gravity gradiometry surveys, or combinations thereof for the purposes of exploration for geologic hydrogen, construction of geologic hydrogen exploration models, or the identification of target zones for carbon sequestration by subsurface carbon mineralization or sulfur sequestration by subsurface sulfur mineralization processes.

The systems and methods disclosed herein relate to the development of methods and systems for identifying, evaluating, and high-grading source rocks associated with the generation of geologic hydrogen using high-resolution measurements of magnetic susceptibility and other rock or mineralogical qualities (e.g., mineral composition, density) to calibrate, integrate, and improve the development of geophysical models and subsurface interpretations. Such systems and methods also provide for the integration of seismic (e.g., reflection, refraction, tomography, ambient noise tomography), airborne magnetic, gravity, and/or gravity gradiometry surveys, or ground-based or sea-based magnetic and gravity surveys, for the purposes of exploration for geologic hydrogen. The systems and methods disclosed herein also provide for constructing geologic hydrogen exploration models and/or using aeromagnetic and aerogravity gradiometry data, or ground-based or sea-based magnetic and gravity data, to identify subsurface lithologic target intervals for hydrogen generation and/or accumulation.

Further, the systems and methods described herein allow for the use of aeromagnetic and aerogravity and gradiometry data, or ground-based or sea-based magnetic and gravity data, to identify and high-grade targets for geothermal energy exploration. Such systems and methods can likewise allow for the use of aeromagnetic and aerogravity and gradiometry data, or ground-based or sea-based magnetic and gravity data, to identify and high-grade exploration targets for the mining of various ore deposits, including critical mineral mining, iron ore mining, or iron oxide-copper-gold (IOCG) mining.

Some examples are specific to the exploration for geologic hydrogen extraction, geothermal energy extraction, or helium extraction present in the subsurface and extracted through the drilling of a well or borehole, flowing fluids to and producing fluids at the surface, or identifying targets for the injection of various fluids into the subsurface for geologic hydrogen stimulation or carbon or sulfur mineralization targets.

Some embodiments include devices and processes for identifying geological hydrogen source rocks, geologic settings, orientations, or structures of geological hydrogen source rocks, key stratigraphic intervals or zones, or intervals where hydrogen has been generated or may be generated or accumulated, and devices and processes for providing data processing and interpretation methods for ground-based, sea-based, or airborne geophysical exploration tools (e.g., reflection/refraction seismology, ambient noise tomography, tomography, magnetometry, gravimetry, gravity gradiometry) based on the use of magnetic susceptibility measurements conducted on rocks or materials collected at the surface, through subsurface drilling (e.g., cuttings, core), or measured through downhole logging for the purposes of exploration for geologic hydrogen.

In the embodiments described herein, magnetic susceptibility measurements are used to identify, evaluate, and high-grade source rocks associated with the generation of geologic hydrogen. Elevated magnetic susceptibility values associated with hydrogen source rock minerals distinguish these rocks from other crustal rocks (e.g., granites, sandstones, carbonates). Alteration reactions that generate hydrogen from iron-rich (dominantly Fe²⁺) source rocks produce minerals with even higher magnetic susceptibility, defining a link between elevated magnetic susceptibility and hydrogen generation. These minerals can be categorized based on both their role (as products or reactants) in hydrogen generation and the type of magnetism they exhibit (ferromagnetism, antiferromagnetism, ferrimagnetism, paramagnetism, or diamagnetism).

Rocks exhibit magnetic susceptibility values that depend on their constituent minerals and may indicate a degree of alteration. These values can be measured from various geologic materials, including drill cuttings, whole/rotary sidewall core, or outcrop samples to define magnetic signatures for unaltered hydrogen source rock (e.g., basalt, gabbro, dunite) and altered lithologies (e.g., serpentinite). Magnetic susceptibilities can serve as proxies for alteration profiles with depth to identify and define intervals that indicate varying degrees of hydrogen source rock maturation (i.e., the proportion of unaltered (“immature”) or altered (“mature”) hydrogen generating mineral phases). These magnetic susceptibility profiles can also be used to create magnetic well “ties” to search for related signals in magnetic or seismic models and extrapolate a feature of interest across a broader geographic or regional scale. High resolution magnetic susceptibility values from multiple boreholes support broader regional geophysical models (e.g., 2D/3D seismic, ambient noise tomography, seismic tomography, AGM) to calibrate and/or improve resolution.

Mineralogy and chemical composition, particularly iron content and oxidation state (e.g., ferrous iron (Fe²⁺) versus ferric iron (Fe³⁺) contained in minerals), may be related to the hydrogen generation processes (i.e., natural geologic hydrogen exploration) and future hydrogen generation potential (i.e., engineered/stimulated processes). These parameters may impact the magnetic susceptibility of rock and can be measured in the laboratory or field using tools such as x-ray fluorescence (XRF) for elemental composition, x-ray diffraction (XRD) for mineralogy, and Mössbauer spectroscopy for iron oxidation state. Magnetic susceptibility measurements can be made using a variety of instrumentation or modelled from sea-based, ground-based, or airborne surveys.

Incorporation of magnetic susceptibility measurements into geologic hydrogen exploration allows for identifying target lithologies to serve as a hydrogen source rock in the natural hydrogen system. The magnetic susceptibility measurements of lithologies associated with past hydrogen generation or potential future hydrogen generation through engineered methods can be scored and used to rank, high-grade and develop geologic hydrogen exploration strategies, and inform drilling decisions by prioritizing areas of interest, high-grading “sweet spots” within target exploration areas. These same approaches are explicitly tied to detecting subsurface targets for carbon dioxide or dihydrogen sulfide gas mineralization.

An embodiment of this disclosure is a method for using measurements of magnetic susceptibility and modelling to identify target zones for natural hydrogen exploration. This may involve collecting magnetic susceptibility from a plurality of subsurface locations within a region, receiving airborne geophysical survey data of that region, tying magnetic susceptibility data of the plurality of subsurface locations with the airborne geophysical data, and generating a geologic map of igneous (mafic or ultramafic) rock, metamorphic rock, or mafic mineral- or iron-rich sedimentary rock within a region based on the tied magnetic susceptibility data using a model. The geophysical survey data may include ground-based, sea-based, or airborne gravity or magnetic data.

In this embodiment, the plurality of subsurface locations may include data that extends vertically (e.g., across depth of a borehole or well or from the surface to subsurface), and the magnetic susceptibility data may include a magnetic anomaly above, below, or within igneous rock, metamorphic rock, or mafic mineral-or iron-rich sedimentary rock. Further, methods of collecting magnetic susceptibility include drilling a borehole and measuring magnetic susceptibility at locations within the borehole.

In some embodiments, magnetic susceptibility of geologic materials may be measured using a vibrating sample magnetometer, a superconducting quantum interference device magnetometer, a magnetic property measurement system, an AC susceptometer, a Kappabridge, a Faraday balance, a Gouy balance, an alternating gradient magnetometer or a wireline magnetic susceptibility tool. Ground-based, sea-based, or airborne gravity and magnetic data may be measured using a gravitometer or gradiometer, and a scalar magnetometer, a vector magnetometer, or a magnetic gradiometer, respectively.

An embodiment of this disclosure includes a method for evaluating geological hydrogen source rock to identify target zones for exploration of natural hydrogen accumulations. This embodiment may include using magnetic susceptibility data from a plurality of surface or subsurface locations, tying magnetic susceptibility data to geophysical survey data, and using statistical models to generate geologic maps identifying zones for hydrogen production or a potential hydrogen accumulation. This embodiment may include generating geologic maps that identify zones for hydrogen production or a potential hydrogen accumulation in a first region, and identifying, using a statistical model, a second region including a target zone of the geological hydrogen source rock having a potential for hydrogen production or a potential for hydrogen accumulation, wherein the second region exhibits above-ground or subsurface characteristics corresponding to the above-ground or subsurface characteristics of the geologic map of the first region.

Other embodiments include a method for evaluating geological hydrogen source rock to identify target zones for exploration of natural hydrogen accumulations without the use of models. This may include the measurement of magnetic susceptibility subsurface locations including along the length of a borehole. This embodiment may use magnetic susceptibility to identify hydrogen source rock alteration and potential zones of hydrogen production or accumulation. This embodiment may also utilize x-ray diffraction, x-ray fluorescence, scanning electron microscopy, dispersive x-ray spectroscopy, or wireline-based rock physical and chemical data. These methods that include identifying a target zone capable of producing hydrogen gas also include hydrogen production exhibiting a carbon intensity score less than 3.0 kg CO₂eq/kg H₂, less than 1.5 kg CO₂eq/kg H₂, or less than 0.45 kg CO₂eq/kg H₂.

In some embodiments, magnetic susceptibility data from locations is tied to geophysical survey data to build improved statistical models that may be used to generate geologic maps. This method involves the measurement of magnetic susceptibility, the collection of ground-based, sea-based, or airborne geophysical survey data, and tying the magnetic susceptibility data to the geophysical survey data using statistical models. In this embodiment, these models may involve Werner Deconvolution forward or inverse modelling. The geologic maps may also integrate other data types including gravity data, electromagnetic data, seismic data, other borehole wireline log data, or mineralogy data or other rock geophysical or geochemical data to generate new geologic maps.

In some embodiments, a method for evaluating geological hydrogen source rock in a region is provided. The method includes: collecting magnetic susceptibility data from a plurality of subsurface locations in the region; receiving geophysical survey data of the region, wherein the geophysical survey data includes geophysical data collected from above ground; tying the magnetic susceptibility data of the plurality of subsurface locations to the geophysical survey data; generating a geologic map of igneous rock, metamorphic rock, or mafic mineral-or iron-rich sedimentary rock within the region based on the magnetic susceptibility data using a model, wherein the model is trained on the geophysical survey data tied to the magnetic susceptibility data; and determining, based on the geologic map, a target zone of the geological hydrogen source rock with a potential for hydrogen production without injection of a reactant into the igneous rock, metamorphic rock, or mafic mineral-or iron-rich sedimentary or with the potential for hydrogen accumulation.

In some aspects, the plurality of subsurface locations includes subsurface locations spanning a vertical axis, and wherein the magnetic susceptibility data includes a magnetic anomaly at a subsurface location above, below, or within the igneous rock, metamorphic rock, or mafic mineral-or iron-rich sedimentary rock within the region. In some aspects, the step of collecting the magnetic susceptibility data includes: drilling a borehole, wherein the plurality of subsurface locations is located within the borehole; and measuring a magnetic susceptibility at each subsurface location. In some aspects, the geophysical survey data includes a measurement of the magnetic field at or above a surface location. In some aspects, the magnetic field is measured using one or more of a scalar magnetometer, a vector magnetometer, or a magnetic gradiometer. In some aspects, the step of collecting the magnetic susceptibility data includes: drilling a borehole, wherein the plurality of subsurface locations is located within the borehole; obtaining a geological sample at each subsurface location; and measuring a magnetic susceptibility of each geological sample.

In some aspects, the magnetic susceptibility is measured using a vibrating sample magnetometer, a superconducting quantum interference device magnetometer, a magnetic property measurement systems, an AC susceptometer, a Kappabridge, a Faraday balance, a Gouy balance, an alternating gradient magnetometer, or a wireline magnetic susceptibility tool. In some aspects, the geophysical survey data includes historical measurements.

In some aspects, the model is a statistical model. In some aspects, the model includes a Werner deconvolution inverse model or a Werner deconvolution forward model. In other embodiments, the geophysical survey data includes airborne magnetic field measurements derived from airborne magnetic surveys or electromagnetic surveys. In some aspects, the geophysical survey data includes gravity data.

In some aspects, the gravity data is provided from airborne gravity or gravity gradiometers, airborne gravity and magnetic surveys, ground-based gravity and magnetic surveys, sea-based gravity and magnetic surveys, or electromagnetic surveys. In some aspects, the geophysical survey data includes seismic data. In some aspects, the seismic data includes one of two-dimensional or three-dimensional seismic reflection or seismic refraction, seismic tomography, or ambient noise tomography data.

In some aspects, the method further includes determining a mineralogy of each geological sample. In some aspects, the mineralogy is determined by x-ray diffraction, x-ray fluorescence, scanning electron microscopy, or scanning electron microscope-energy dispersive x-ray spectroscopy. In some aspects, the mineralogy is normative mineralogy.

In some aspects, the techniques described herein relate to a method, wherein the target zone is a target zone of potential hydrogen production, and wherein the step of determining the target zone includes identifying lithological zones corresponding to source rock capable of generating hydrogen. In some aspects, the target zone is a target zone of potential hydrogen accumulation, and the step of determining the target zone includes identifying lithological zones corresponding to source rock capable of serving as a reservoir for hydrogen accumulation.

In some aspects, the method further includes identifying lithological zones corresponding to source rock capable of providing hydrogen, hydrogen derivatives, carbon mineralization, or sulfur mineralization. In some aspects, the target zone is capable of producing a hydrogen gas product exhibiting a carbon intensity score less than 3.0 kg CO₂eq/kg H₂. In some aspects, the hydrogen gas product exhibits a carbon intensity score less than 1.5 kg CO₂eq/kg H₂. In some aspects, the hydrogen gas product exhibits a carbon intensity score less than 0.45 kg CO₂eq/kg H₂.

In some aspects, a method for evaluating geological hydrogen source rock is provided. The method includes: receiving a training dataset including magnetic susceptibility data from a plurality of subsurface locations in a first region and survey data of the first region, wherein the survey data includes geophysical survey data collected from above ground, wherein the survey data is calibrated to the magnetic susceptibility data of the plurality of subsurface locations through well ties; training, using the training dataset, a statistical model to generate a geologic map of the first region, wherein the geologic map of the first region includes a target zone of the geological hydrogen source rock with a potential for hydrogen production without use of a reactant, wherein the geologic map of the first region includes above-ground or subsurface characteristics; and identifying, using the statistical model, a second region including a target zone of the geological hydrogen source rock having a potential for hydrogen production or a potential for hydrogen accumulation, wherein the second region exhibits above-ground or subsurface characteristics corresponding to the above-ground or subsurface characteristics of the geologic map of the first region.

In some aspects, the above-ground characteristics of the geologic map of the first region include ground-based, sea-based or airborne gravity and magnetic data. In some aspects, the plurality of subsurface locations includes locations within igneous rock, metamorphic rock, or mafic mineral-or iron-rich sedimentary rock in the region. In some aspects, the plurality of subsurface locations includes subsurface locations spanning a vertical axis, and wherein the magnetic susceptibility data includes a magnetic anomaly at a subsurface location above, below, or within the igneous rock, metamorphic rock, or mafic mineral-or iron-rich sedimentary rock within the region.

In some aspects, the target zone is capable of producing a hydrogen gas product exhibiting a carbon intensity score less than 3.0 kg CO₂eq/kg H₂. In some aspects, the hydrogen gas product exhibits a carbon intensity score less than 1.5 kg CO₂eq/kg H₂. In some aspects, the hydrogen gas product exhibits a carbon intensity score less than 0.45 kg CO₂eq/kg H₂.

In some embodiments, a method for evaluating geological hydrogen source rock in a region is provided. The method includes: obtaining a plurality of geological samples of igneous rock, metamorphic rock, or mafic mineral-or iron-rich sedimentary rock within the geological hydrogen source rock of the region; measuring a magnetic susceptibility corresponding to each geological sample; obtaining geophysical survey data of the region; and determining, based on the plurality of magnetic susceptibilities and the geophysical survey data, a target zone of the geological hydrogen source rock having a potential of hydrogen production without use of a reactant or a potential of hydrogen accumulation.

In some aspects, the step of measuring includes measuring the magnetic susceptibility of each geological sample using a vibrating sample magnetometer, a superconducting quantum interference device magnetometer, a magnetic property measurement systems, an AC susceptometer, a Kappabridge, a Faraday balance, a Gouy balance, an alternating gradient magnetometer, or a wireline magnetic susceptibility tool. In some aspects, the geophysical survey data includes measuring the field at a above a surface location.

In some aspects, the magnetic field is collected using one of a scalar magnetometer, a vector magnetometer, or a magnetic gradiometer.

In some aspects, the method further includes determining a mineralogy of each geological sample. In some aspects, the mineralogy is determined by x-ray diffraction, x-ray fluorescence, scanning electron microscopy, or scanning electron microscope-energy dispersive x-ray spectroscopy. In some aspects, the mineralogy is normative mineralogy.

In some aspects, the method further includes identifying a zone of sealing lithology for a zone of hydrogen accumulation. In some aspects, the step of identifying the zone of sealing lithology includes identifying one or more geological samples having a low magnetic susceptibility and a mineralogy including a mineral capable of generating hydrogen or sequestering carbon or sulfur through mineralization. In some aspects, the plurality of geological samples includes one or more geological samples taken at a plurality of elevations at a location in the region. In some aspects, the plurality of geological samples includes one or more geological samples taken at a plurality of locations at a consistent elevation within the region. In some aspects, the method further includes determining relative changes in magnetic susceptibility. In some aspects, the method further includes determining a degree of mineral alteration associated with hydrogen generation and a porosity of the geological sample. In some aspects, the porosity includes one of matrix porosity and fracture porosity. In some aspects, the porosity is measured using density porosity, neutron porosity, far-field sonic, or nuclear magnetic resonance.

In some aspects, the degree of mineral alteration includes a low degree of alteration indicated by a low magnetic susceptibility or a high degree of alteration indicated by a high magnetic susceptibility. In some aspects, the techniques described herein relate to a method, wherein the target zone is a target zone of the geological hydrogen source rock having the potential of hydrogen accumulation. In some aspects, the geophysical survey data includes one or more of mineralogical data, magnetic field data, magnetic field gradient data, gravitational data, gravitational gradient data, spatially resolved well data, wireline data, wireline instrumentation data, wireline density data, acoustic impedance data, seismic data, or electromagnetic data.

In some aspects, the method further includes mapping the magnetic susceptibilities of the plurality of geological samples and the geophysical survey data to identify the target zone of potential hydrogen production or potential hydrogen accumulation. In some aspects, the step of mapping includes identifying continuous boundaries of target zones. In some aspects, the target zones include in situ generation of hydrogen and/or other hydrogen derivatives.

In some aspects, the step of determining the target zone includes: identifying a plurality of magnetic well ties between the magnetic susceptibility data and the geophysical survey data; and determining, from the plurality of magnetic well ties, the target zone of potential hydrogen generation or potential hydrogen accumulation. In some aspects, the target zone of potential hydrogen production or potential hydrogen accumulation is a target zone of potential hydrogen production without use of the reactant. In some aspects, the target zone of potential hydrogen production or potential hydrogen accumulation is a target zone of potential hydrogen accumulation.

In some aspects, the target zone is capable of producing a hydrogen gas product exhibiting a carbon intensity score less than 3.0 kg CO₂eq/kg H₂. In some aspects, the hydrogen gas product exhibits a carbon intensity score of less than 1.5 kg CO₂eq/kg H₂. In some aspects, the hydrogen gas product exhibits a carbon intensity score of less than 0.45 CO₂eq/kg H₂.

In some aspects, a method for measuring magnetic susceptibility of geological hydrogen source rock at a region is provided. The method including: collecting magnetic susceptibility data from a plurality of subsurface locations in the region, wherein the plurality of subsurface locations includes subsurface locations in igneous rock, metamorphic rock, or mafic mineral-or iron-rich sedimentary rock within the region, wherein the magnetic susceptibility data includes a magnetic anomaly at a subsurface location above, below, or within the igneous rock, metamorphic rock, or mafic mineral- or iron-rich sedimentary rock, wherein the igneous rock, metamorphic rock, or mafic mineral- or iron-rich sedimentary rock includes a target zone of the geological hydrogen source rock having a potential of hydrogen production without injection of a reactant or a potential of hydrogen accumulation; receiving geophysical survey data of the region, wherein the geophysical survey data includes geophysical data collected above ground; and tying the magnetic susceptibility data to the geophysical survey data.

In some aspects, the step of collecting magnetic susceptibility data includes measuring magnetic susceptibility at the plurality of subsurface locations using a vibrating sample magnetometer, a superconducting quantum interference device magnetometer, a magnetic property measurement systems, an AC susceptometer, a Kappabridge, a Faraday balance, a Gouy balance, an alternating gradient magnetometer, or a wireline magnetic susceptibility tool.

In some aspects, the step of collecting further includes measuring the magnetic susceptibility along a depth of the well or borehole. In some aspects, the step of collecting the magnetic susceptibility data includes: obtaining a plurality of geological samples of geological hydrogen source rock within the region, each geologic sample taken at a subsurface location of the plurality of subsurface locations; and measuring a magnetic susceptibility of each geological sample using a laboratory or field-based instrument on the geological sample.

In some aspects, the geophysical survey data includes regional magnetic survey data collected by a ground-based magnetic method, a sea-based magnetic method, or an airborne magnetic method. In some aspects, the magnetic survey uses one or more of a scalar magnetometer, a vector magnetometer, or a magnetic gradiometer. In some aspects, the method further includes generating a map of the region using a statistical model. In some aspects the statistical model includes a Werner deconvolution model. In some aspects, the Werner deconvolution modeling is inverse modeling. In some aspects, the Werner deconvolution modeling is forward modeling.

In some aspects, the method further includes identifying lithologic intervals using the magnetic susceptibility. In some aspects, the techniques described herein relate to a method, further including determining the mineral composition of each geological sample. In some aspects, the techniques described herein relate to a method, further including differentiating rock types.

In some aspects, the method further includes measuring a wireline density at a location from which the geological sample was taken. In some aspects, the geophysical survey data includes one or more of mineralogical data, magnetic field data, magnetic field gradient data, gravitational data, gravitational gradient data, spatially resolved well data, wireline data, wireline instrumentation data, wireline density data, acoustic impedance data, seismic data, or electromagnetic data. In some aspects, the geophysical survey data includes airborne gravity or gravity gradiometers, airborne gravity and magnetics, ground-based gravity and magnetics, or sea-based gravity and magnetics, or electromagnetic surveys integrated with seismic reflection data.

In some aspects, the techniques described herein relate to a method, further including identifying lithological zones corresponding to a target zone of potential hydrogen production or hydrogen accumulation. In some aspects, the target zone is capable of producing a hydrogen gas product exhibiting a carbon intensity score less than 3.0 kg CO₂eq/kg H₂. In some aspects, the hydrogen gas product exhibits a carbon intensity score of less than 1.5 kg CO₂eq/kg H₂. In some aspects, the hydrogen gas product exhibits a carbon intensity score of less than 0.45 kg CO₂eq/kg H₂.

Features from any of the disclosed embodiments may be used in combination with one another, without limitation. In addition, other features and advantages of the present disclosure will become apparent to those of ordinary skill in the art through consideration of the following detailed description and the accompanying drawings.

The foregoing summary is provided merely for the purpose of summarizing some example embodiments described herein. Because the embodiments described above and below are merely examples, they should not be construed to narrow the scope of this disclosure in any way. It will be appreciated that the scope of the present disclosure encompasses many potential embodiments in addition to those summarized above, some of which will be described in further detail below.

### BRIEF DESCRIPTION OF THE FIGURES

Having described certain example embodiments in general terms above, reference will now be made to the accompanying drawings, which are not necessarily drawn to scale. The drawings provided in FIGS. 1-19 illustrate several embodiments of the disclosure, wherein identical reference numerals refer to identical or similar elements or features in different views or embodiments shown in the drawings.

FIG. 1 is a diagram illustrating the range of magnetic susceptibilities for generalized geologic materials.

FIG. 2 is a diagram illustrating the range of bulk densities of generalized geologic materials.

FIG. 3 is a table describing the idealized reactants and products of some hydrogen-generating processes, namely serpentinization and pyritization reactions and carbon-sequestering decarbonation reactions.

FIG. 4 is a table of minerals associated with metamorphism of mafic and ultramafic rocks with respect to increasing grade of metamorphism.

FIG. 5 is a diagram illustrating minerals associated with metamorphism of mafic and ultramafic rocks with respect to increasing grade of metamorphism. Primary hydrogen generating minerals (H₁) are outlined by rectangular boxes and secondary hydrogen generating minerals (H₂) are outlined by ovoidal boxes.

FIG. 6 is a diagram showing the stability of critical minerals in serpentinized rock with respect to metamorphic grade (temperature) and facies.

FIG. 7 is a table of common minerals and their style of magnetism and typical magnetic susceptibilities.

FIG. 8 is a table of minerals categorized based on their role in hydrogen generation.

FIG. 9 is a plot of common metamorphic facies (i.e., mineral assemblages formed across various pressure and temperature pathways conditions) documenting the metamorphic evolution of mafic rocks.

FIG. 10 is a plot of common metamorphic facies (i.e., mineral assemblages formed across various pressure and temperature pathways conditions) that evolve during low-pressure metamorphism of mafic rocks across a range of temperatures.

FIG. 11 is a diagram illustrating the typical mineral assemblage of peridotites based on temperature and pressure regimes during crystallization.

FIGS. 12-14 illustrate example methods for evaluating geological hydrogen source rock in a region using high-resolution magnetic susceptibility measurements and geophysical survey data to identify lithological zones of interest and/or natural hydrogen accumulations.

FIG. 15 is a block diagram that describes the process of measuring magnetic susceptibility in samples.

FIG. 16 is a workflow diagram that describes the processing of regional airborne gravity and magnetic data using magnetic susceptibility measurements for enhanced geologic modelling.

FIG. 17A is a diagram that illustrates general trends of seismic velocities through ultramafic rocks as related to the degree of serpentinization.

FIG. 17B is a diagram illustrating the relationship between density and magnetic susceptibility with increasing serpentinization of ultramafic rocks.

FIG. 18 is a workflow diagram that describes the process of integrating magnetic susceptibility measurements, enhanced geologic models from regional airborne gravity and magnetic data, and seismic data to improve identification of hydrogen-related lithologies.

FIG. 19 illustrates an example for utilizing magnetic susceptibility data combined with geophysical survey data in one region to identify geological hydrogen source rock in other regions.

### DETAILED DESCRIPTION

Some example embodiments will now be described more fully hereinafter with reference to the accompanying figures, in which some, but not necessarily all, embodiments are shown. Because inventions described herein may be embodied in many different forms, the invention should not be limited solely to the embodiments set forth herein; rather, these embodiments are provided so that this disclosure will satisfy applicable legal requirements.

As used herein, and unless the context dictates otherwise, the following terms have the meanings as specified below.

The term “geologic hydrogen” generally refers to hydrogen produced from a geological formation.

The term “geologic hydrogen source” generally refers to any source of geologic hydrogen, such as a wellhead connected to a wellbore or any other pathway by which geologic hydrogen may be transmitted.

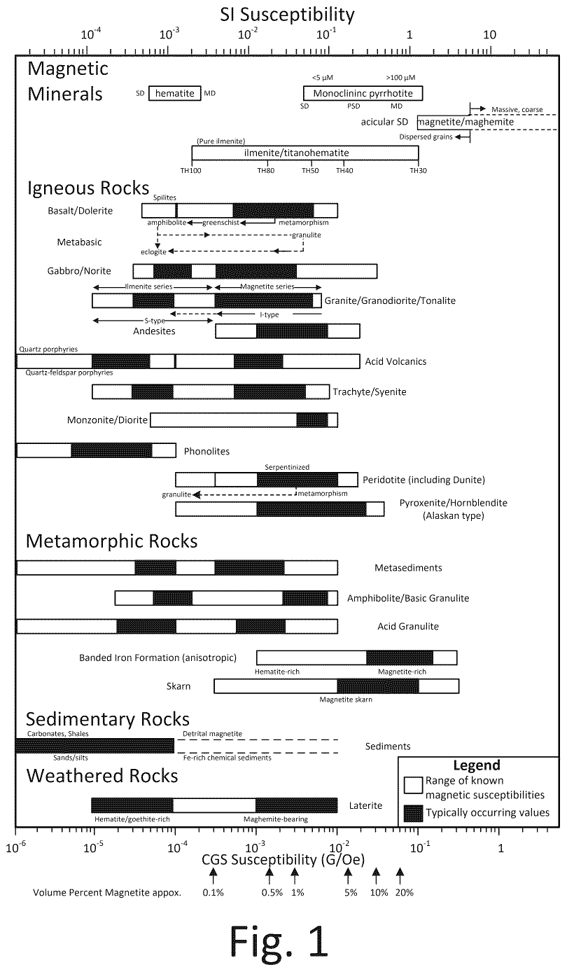

FIGS. 1-19 illustrate details and aspects of the disclosure related to exemplary methods of evaluating a geological hydrogen source rock at various scales (e.g., cuttings, cores, borehole, drill site, prospect, geologic province). Embodiments disclosed herein include systems and methods of analyzing rock or sediment recovered from the drilling of a well or borehole, or rock or sediment in contact with a drilled well or borehole (i.e., rock that comprises the well or borehole walls), and rock or sediment collected from the surface. Another set of embodiments focuses on incorporating the data into geologic hydrogen exploration efforts, the identification of geologic materials that can generate or have previously generated hydrogen, and the identification and mapping of geologic materials favorable for hydrogen accumulation in either hydrogen source rock reservoirs (unconventional) or natural hydrogen reservoirs (conventional). For example, reservoir and/or sealing formations (e.g., basal sandstone reservoir rich in iron oxide minerals lying above mafic crystalline basement, an igneous sill acting as a seal in a stacked sequence of igneous intrusions) overlying hydrogen source rock may be mappable using regional magnetic field data that are integrated with magnetic susceptibility measurements directly measured from a borehole. Embodiments disclosed additionally allow for the processing and interpretation of data gathered using ground-based, sea-based, or airborne geophysical exploration tools (e.g., reflection or refraction seismology, tomography, ambient noise tomography, magnetometry, gravimetry, gravity gradiometry) for the purposes of exploration for geologic hydrogen. In some embodiments, the systems and methodologies involve the analysis of geophysical, geochemical, and mineralogical characteristics of geologic materials for use in geologic hydrogen exploration.

It is understood that the techniques disclosed herein are not so limited and find application in the drilling for a variety of naturally occurring molecules, including hydrogen, dihydrogen sulfide, hydrogen derivatives, helium, other noble gases, hydrocarbons, nitrogen, and carbon dioxide, geothermal heat, and ore deposits of various types.

It is also understood that these techniques may include drilling for the recovery of other natural subsurface resources (e.g., geothermal heat, minerals/ores, groundwater), and the production, purification, or handling of those resources, drilling for the purpose of subsurface sequestration of fluids (e.g., carbon dioxide, dihydrogen sulfide), gas storage (e.g., hydrocarbon, hydrogen, or helium), brine or wastewater disposal, enhanced geothermal, and other types of drilling into the subsurface where fluids may be detected, monitored, or quantified.

In some embodiments, the present disclosure involves the utilization of magnetic susceptibility of a rock or sediment, which is a quantitative measurement of the extent to which a material may be magnetized in relation to a given applied magnetic field. As part of the embodiments disclosed herein, magnetic susceptibility measurements can be used alone or in combination with other measurements such as mineralogy, geochemistry, and other geophysical measurements (e.g., gravity, seismic, other wireline logging techniques). Magnetic susceptibility can be measured on rocks or drill cuttings collected within a well or borehole, sediments, split rock core, whole core, sidewall core, cored outcrop, or subcrop.

In all cases, these measurements may enable the identification of geologic settings favorable for hydrogen generation or accumulation and hence can be used to identify exploration targets for natural hydrogen or to identify targets for carbon or sulfur sequestration by mineralization. The high-resolution magnetic susceptibility and rock property data can also be used for the processing and interpretation of data gathered using ground-based, sea-based, or airborne geophysical exploration tools (e.g., reflection/refraction seismology, tomography, ambient noise tomography, magnetometry, gravimetry, gravity gradiometry) for the purposes of exploration for geologic hydrogen by conventional or unconventional methods. Magnetic susceptibility and geologic processes influencing magnetic susceptibility and geologic hydrogen are hereafter described in more detail.

### Importance of Source Rock Evaluation in Geologic Hydrogen Systems Analysis

Hydrogen systems exploration workflows involve the identification and mapping of suitable quality geological hydrogen source rocks that contain elevated proportions of minerals involved in the generation of hydrogen in both unaltered (primary) and altered (secondary) states that can be associated with elevated magnetic susceptibility. Natural hydrogen exploration involves determining a lithology or the parts of a lithology, basins, blocks, or other geologic/geographic features that contain suitable quality source rocks that have sufficient rates of hydrogen generation on a moles of hydrogen per mass or volume of rock basis to yield hydrogen accumulation or “sweet spots.”

Identifying the optimal locations to find hydrogen generating source rocks depends upon the natural hydrogen exploration strategy. “Conventional” exploration seeks to identify natural hydrogen that formed and migrated to and accumulated within reservoir rocks contained by lithological seals with suitable trapping geometries. By comparison, “unconventional” exploration seeks to identify natural hydrogen that formed and charged source-reservoir systems and is dominantly contained within mature source rocks. Each exploration pathway requires the identification of mature source rocks that have generated significant volumes of hydrogen, with a priority on quantifying optimal zones of hydrogen generation and allowing for quantitative assessment of the relative volumes of hydrogen generation with respect to the size of prospective reservoirs. Further, both conventional and unconventional are referred to as natural hydrogen generation, meaning hydrogen production or generation that occurs without the injection of a reactant or stimulant into a geological source rock.

“Conventional” natural hydrogen exploration processes involve identification of high-quality source rocks that have previously generated significant volumes of hydrogen that would have migrated through subsurface formations and potentially accumulated within reservoirs contained within structural or stratigraphic traps and sealed by a low porosity and low permeability lithology. In that scenario, the natural hydrogen source rock has a high initial proportion of primary minerals involved in hydrogen generation without an abundance of other unrelated minerals and has a present mineralogy dominated by altered mineral phases that have already generated hydrogen during the alteration process. As a result, a method to score and rank high-quality source rocks for conventional hydrogen exploration is required in order to develop exploration workflows for conventional natural hydrogen, such as mapping source rocks, migration pathways, prospective reservoirs, prospective traps, and prospective sealing lithologies or develop robust fetch and focus or charge models. Source rock evaluation tools are also used to rank prospective hydrogen systems at various scales, such as globally, within a particular region, within a country, within a basin or system, within a lithology or sub-lithology, or even within a borehole.

“Unconventional” natural hydrogen exploration processes involve identification of high-quality source rocks that have generated significant volumes of hydrogen and either have accumulated hydrogen within source rock-reservoirs or are actively generating hydrogen at economic rates and volumes. Exploration for source rock-reservoirs is in some ways analogous to exploration for shales that are both source rocks and serve as unconventional reservoirs for hydrocarbon extraction. As a result, a method to score and rank high-quality source rocks for unconventional hydrogen exploration involves identifying specific targets for unconventional natural hydrogen exploration in these settings. Robust source rock evaluation tools may also be used to rank prospective unconventional hydrogen systems at various scales, such as globally, within a particular lithology or sub-lithology, a region, within a country, within a basin or system, or even within a borehole.

Exploration for stimulated or enhanced hydrogen production (EHP) involves the identification of high-quality source rocks that can be altered by engineering processes to catalyze active generation of significant volumes of hydrogen, while potentially also permanently sequestering carbon or sulfur through mineralization. As a result, a method to score and rank high-quality source rocks should be developed in order to identify specific targets for enhanced hydrogen production scenarios. In contrast to natural hydrogen exploration or production, enhanced hydrogen production (EHP) requires the injection of a reactant or stimulant into a geological source rock for hydrogen production or generation.

The focus of this disclosure describes the detection of geological hydrogen source rocks that been altered to generate significant volumes of hydrogen (based on mineralogy) that can accumulate in either conventional or unconventional reservoirs. The methods described above and below detail the systems and methods of using measurements of magnetic susceptibility and associating them with mineralogy and geologic processes involved in hydrogen generation to evaluate natural hydrogen systems. These concepts and data can advance the understanding of hydrogen systems, especially regarding the characterization and detection of sources, reservoirs, and seals, and improve geologic hydrogen exploration strategies.

### Geologic Hydrogen Generation and Source Rocks

In some embodiments, the most prevalent high-quality source rock for natural hydrogen systems consists of iron-rich rock, iron-rich mafic or ultramafic rock (e.g., basalt, gabbro, diabase, peridotite, dolerite, dunite, harzburgite, lherzolite) with large proportions of iron in the reduced form of ferrous iron (Fe²⁺), or less commonly metallic iron (Fe⁰), or other rocks with large proportions of minerals containing iron in the reduced form (Fe²⁺, or less commonly Fe⁰), including metamorphosed or hydrothermally altered mafic or ultramafic igneous rock, olivine-or pyroxene-bearing metamorphic or sedimentary rock or sediment, metamorphosed or hydrothermally altered olivine-or pyroxene-bearing metamorphic or sedimentary rock or sediment, serpentine mineral-bearing rock or sediment, partially or completely serpentinized rock, serpentinite, eclogites, prehnite-or pumpellyite-rich rock, amphibole-rich igneous or metamorphic rock, amphibolite, pyrite-bearing rock, or an iron-rich or other metalliferous ore deposit. Generally, high-quality source rock for hydrogen generation is found in the igneous and/or metamorphic rock which can extend up to the earth's surface but is often located below the sedimentary basin within an area or region. In some settings, high-quality source rocks may occur at intermediate depths in the subsurface, or be interlayered with sedimentary rocks (i.e., as igneous dikes or sills or intermittent episodes of lava flows) or other igneous or metamorphic rocks. Hydrogen source rocks may even include sedimentary rocks rich in mafic minerals (e.g., olivine, pyroxene) or other iron-rich (e.g., magnetite or hematite) minerals (e.g., basaltic sandstones or banded iron formations).

Optimal source rocks for natural or geologic hydrogen generation originally contained abundant amounts of ferrous iron (Fe²⁺), or reduced iron. Reduced iron is typically observed within minerals such as olivine (e.g., fayalite), orthopyroxene (e.g., ferrosilite), clinopyroxene (e.g., hedenbergite), magnetite, and iron-rich amphibole. Natural hydrogen is formed during the oxidative alteration of these reduced forms of iron. Each of these potential hydrogen source minerals displays significant variation in magnetic susceptibility during initial emplacement. As a result, rocks that contain variable mixtures of common mafic minerals (e.g., olivine, orthopyroxene, clinopyroxene, plagioclase, magnetite, and iron-rich amphibole) can display heterogeneity in magnetic susceptibility, resulting from differences in mineral and magmatic composition as well as in their modes of emplacement.

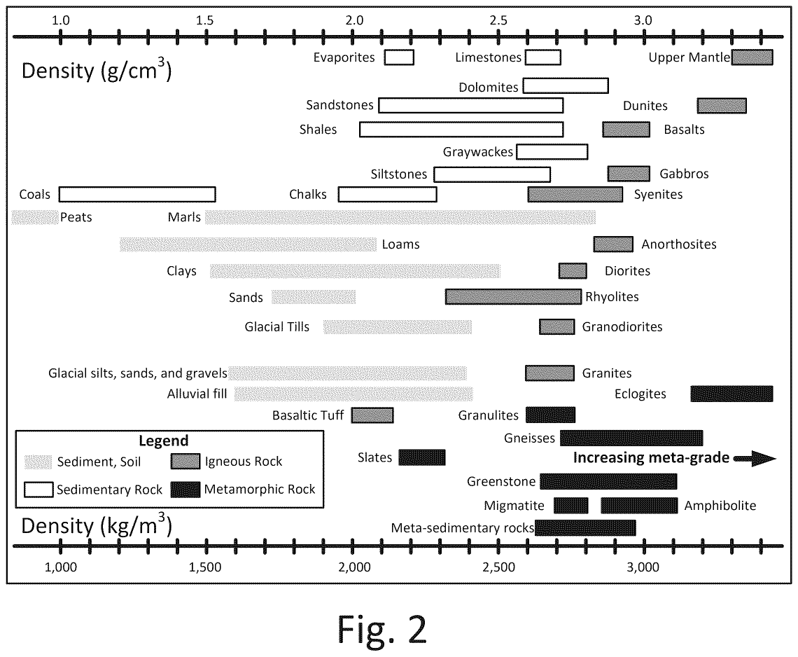

In particular, mafic rocks, such as basalt and gabbro, and ultramafic rocks, such as ferrobasalt, peridotite, dunite, harzburgite, and lherzolite, contain relatively elevated and high proportions, respectively, of ferromagnesian minerals like olivine and pyroxene and minor amounts of iron oxide minerals such as magnetite, hematite, and ilmenite following initial emplacement of mafic/ultramafic magmatic materials. As a result, referring to FIG. 1, mafic and ultramafic rocks display relatively high magnetic susceptibility when compared to typical crustal rocks, such as quartz- and alkali feldspars-rich igneous rocks, such as granites, or sedimentary rocks, such as sandstones, carbonates, or shales. Additionally, the mafic/ultramafic lithologies include minerals with higher densities compared to minerals more typically found in crustal rocks, like quartz-and alkali feldspars-rich igneous rocks, such as granites, or sedimentary rocks, such as sandstones, carbonates, or shales, as shown in FIG. 2.

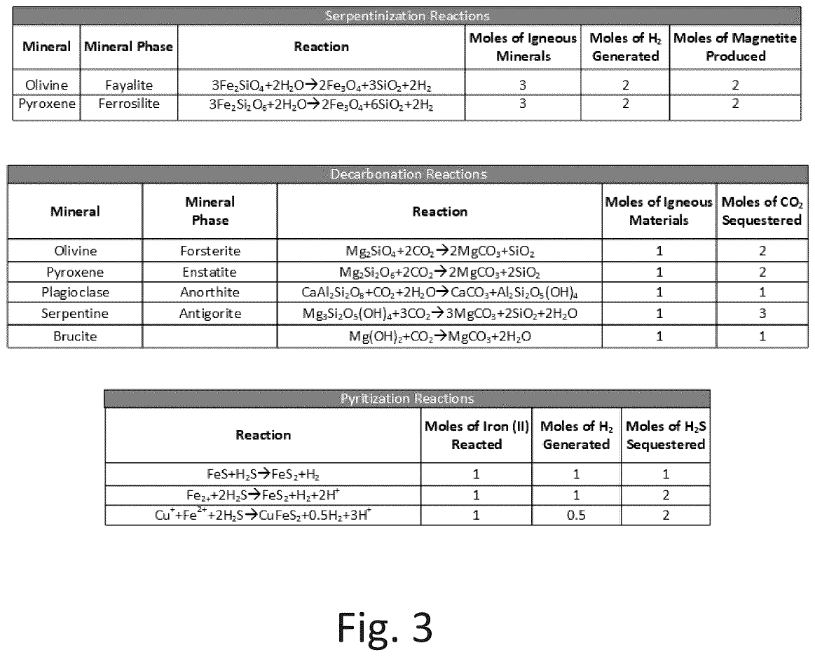

As these natural hydrogen source rocks undergo alteration through water-rock interactions, hydration, hydrothermal alteration, or metamorphism, the reduced forms of iron (Fe²⁺, or less commonly Fe⁰), contained in various iron-rich mineral assemblages are oxidized to ferric iron (Fe³⁺) and behave as an electron donor that can react with other chemical species in the subsurface. These alteration processes may generate Fe³⁺ that is incorporated into secondary mineral products. In a variety of subsurface environments, the free electron created by these processes (e.g., during serpentinization) can interact with and reduce water (i.e., lower the oxygen fugacity) to produce hydrogen gas (H₂) according to idealized reactions shown in FIG. 3. When the alteration reactions described above deplete electron acceptors from pore fluids, thermodynamically stable (or metastable) mineral phases will incorporate Fe³⁺ and the reduction of water (i.e., removal of oxygen) will form hydrogen gas.

Alteration of olivine, orthopyroxene, or clinopyroxene with water or carbon dioxide may lead to the formation of serpentine group minerals (e.g., chrysotile, lizardite, antigorite) and magnetite while releasing hydrogen gas (i.e., serpentinization or serpentinization reactions), as shown in FIG. 3. Additionally, in geologic settings where both minerals containing Fe²⁺ and elevated dihydrogen sulfide or other reactive sulfur compounds are present, such as those often found in geothermal fields associated with volcanic activity, sulfur may react with labile Fe²⁺ (e.g., as a result of the dissolution of olivine, pyroxene, or other minerals) to generate iron sulfide minerals, such as pyrite (FeS₂), while releasing hydrogen gas (see FIG. 3).

Serpentinization and pyritization reactions both release geologic hydrogen gas and generate magnetite (a mineral with relatively high magnetic susceptibility, ˜3 SI units) or pyrite (a mineral with relatively weak magnetic susceptibility, ˜1.5×10⁻³ SI units), respectively. The overall magnetic susceptibility of serpentinized mafic or ultramafic rock therefore may serve as a metric of the degree (or efficiency) of primary alteration (and hence hydrogen generation), increasing as the reactions proceed to generate more magnetite (and hydrogen) from the lower magnetic susceptibility reactants, namely olivine, orthopyroxene, or clinopyroxene. For example, the magnetic susceptibility for unserpentinized peridotites may be as low as ˜2×10⁻³ SI units, whereas the magnetic susceptibility of serpentinized rock containing magnetite has been shown to increase by two orders of magnitude, up to ˜2×10⁻¹ SI units (FIG. 1). In contrast, the magnetic susceptibility of rocks may change relatively immeasurably when pyrite precipitates in instances where the labile Fe²⁺ is sourced from relatively low magnetic susceptibility minerals such as olivine, orthopyroxene, or clinopyroxene (e.g., within the igneous body, within a layered mafic intrusion). However, pyrite may subsequently convert into pyrrhotite, which possesses a much higher magnetic susceptibility (˜0.17-3.20 SI units) if the pyrite-producing formation later undergoes metamorphism (e.g., basalt metamorphosed to greenstone).

The mineral alteration products from these reactions may incorporate variable concentrations of iron or other elements with magnetic properties into secondary minerals with exclusively Fe²⁺, exclusively Fe³⁺, predictable proportions of Fe²⁺ and Fe³⁺, or variable proportions of Fe²⁺ and Fe³⁺. These processes form a variety of secondary mineral phases with increased magnetic susceptibility, raising the magnetic susceptibility of the overall lithology relative to unaltered rock. In some embodiments, therefore, elevated magnetic susceptibility measurements indicate hydrogen-generating alteration reactions, whereas low magnetic susceptibility measurements may indicate little or no comparable alteration.

In comparison to iron-rich mafic rock, some other iron-rich rocks are unlikely to be significant sources of natural hydrogen generation. For example, banded iron formations (BIF) are rich in iron (e.g., elevated magnetite and hematite content) but are deposited following the oxidation of Fe²⁺ to Fe³⁺ in oceans following global oxidation events. Thus, while magnetite in BIFs may constitute additional, low efficiency sources of hydrogen generation, the formation of oxidized iron mineral phases is unlikely to be related to hydrogen formation. Nonetheless, BIFs in some cases may actually have higher magnetic susceptibility than other lithologies related to hydrogen generation. In other scenarios, alteration of BIFs may lead to the generation of maghemite or hematite formation instead, which may exhibit lower magnetic susceptibility; these lithologies may complicate a simplistic deployment of magnetic susceptibility measurements without accounting for additional geologic context. Thus, other sources of data such as total iron content, geologic setting, and other mineralogical or geophysical measurements (e.g., bulk density) can be used to refine the use of magnetic susceptibility.

### Mafic Mineral Alteration and Magnetic Susceptibility

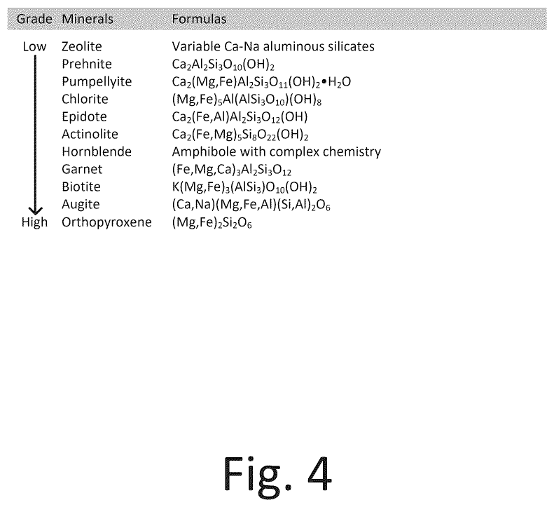

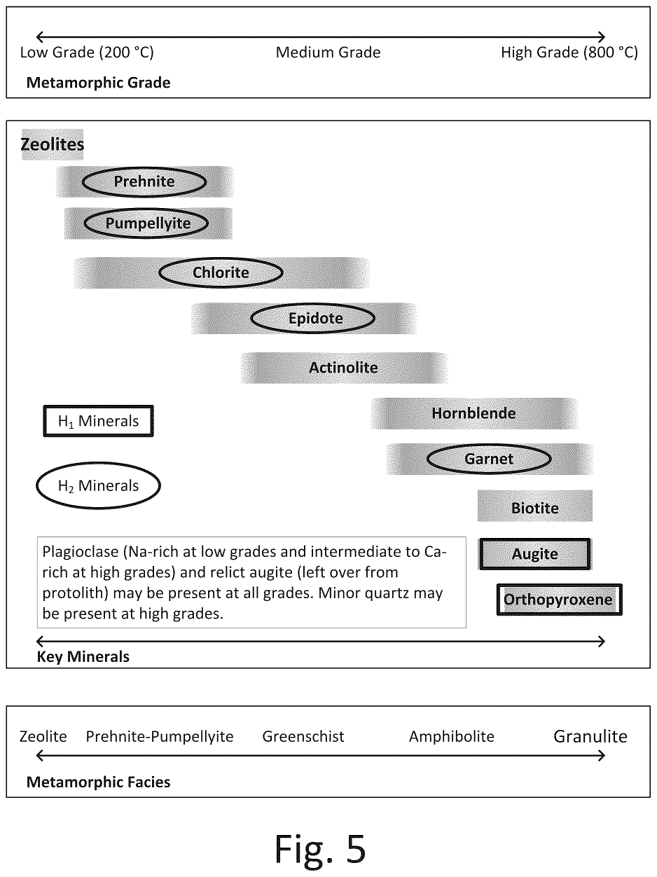

Alteration of common minerals in mafic rock (e.g., olivine, orthopyroxene, or clinopyroxene) can produce hydrogen during various alteration reactions, including water-rock interactions, hydrothermal alteration, or metamorphism of mafic or ultramafic rocks, with the most idealized endmember of the latter resulting in the formation of serpentine. FIGS. 4 and 5 provide minerals associated with metamorphism of mafic and ultramafic rocks with respect to increasing grade of metamorphism. Reactions associated with each mineral alteration pathway display variable hydrogen generation yields and a different consortium of alteration minerals that depend on the pressure and temperature pathways. As a result, the minerals produced in these reactions are indicative tracers of a given rock's geologic pressure-temperature history. In addition to recording the pressure-temperature conditions of chemical alteration, these index minerals typically display predictable quantities of Fe³⁺ (also Fe³⁺/Fe²⁺), which are directly proportional to both the volume of hydrogen generation and magnetic susceptibility. As a result, well-characterized and high-resolution magnetic susceptibility measurements can be important proxies for hydrogen generation.

Other factors such as silica content and temperature can further influence magnetic susceptibility. For example, the proportion of silica in mafic and ultramafic rocks can affect the serpentinization efficiency, hydrogen generation potential, and the magnetic susceptibility of a rock by controlling the proportion and abundance of magnetite generation. In relatively high silica mafic rocks (e.g., basalt), a greater proportion of Fe²⁺ is sequestered into alteration minerals such as chlorite and amphiboles instead of the idealized magnetite endmember following the formation of serpentine minerals; incorporation of Fe²⁺ reduces the efficiency of hydrogen generation from silica-rich mafic/ultramafic rocks, as well as reducing the magnetic susceptibility.

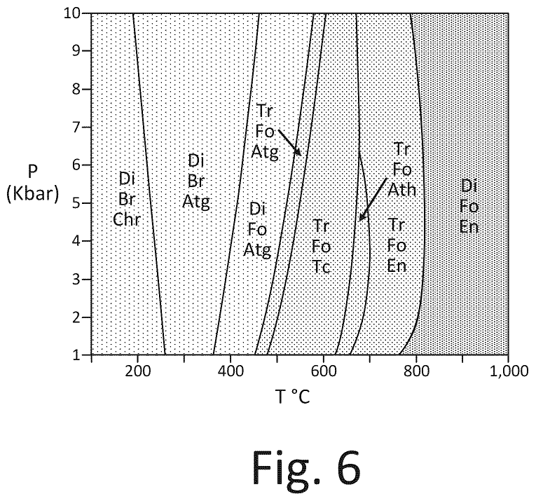

Similarly, the pressure-temperature conditions under which the reaction occurs are also crucial to both the resulting mineral constituents and the magnetic susceptibility of the resulting mineral phases. At the lower range of serpentinization reactions (e.g., <150° C.) there is greater proportion of the mineral brucite (Mg(OH)₂) into which ferrous iron can substitute for the magnesium ion, together with serpentine group minerals (e.g., antigorite, chrysotile, or lizardite), similarly preventing the generation of magnetite and hydrogen, and producing a relatively low magnetic susceptibility mineral phase. At the higher range of temperatures for serpentinization reactions, alternative metamorphic pathways can influence the serpentinization of mafic mineral phases (FIG. 6). For example, at temperatures >315° C., olivine quickly enters into equilibrium with the fluid and alteration minerals, enabling olivine to remain a stable mineral phase under these conditions. As a result, at temperatures >315° C., serpentine formation decreases (i.e., only a portion of the olivine present reacts). Therefore, during higher temperature reactions (>315° C.), alteration of mafic/ultramafic rocks leads to lower amounts of formed serpentine and magnetite, resulting in lower magnetic susceptibility, and less hydrogen being generated per unit mass from the bulk rock.

Thus, the relative proportion of mafic/ultramafic minerals that are altered by various hydration, hydrothermal, and/or metamorphic reactions and both the associated magnetic susceptibility of those minerals and the volumes of hydrogen they have generated are strongly correlated. Well-calibrated magnetic susceptibility models provide an important method capable of identifying zones of geologic hydrogen generation, as well as the importance of properly calibrating magnetic susceptibility measurements when using aeromagnetic data to map key components of the hydrogen system, especially geological hydrogen source rocks.

### Types of Magnetism and Their Characteristics in Geologic Minerals

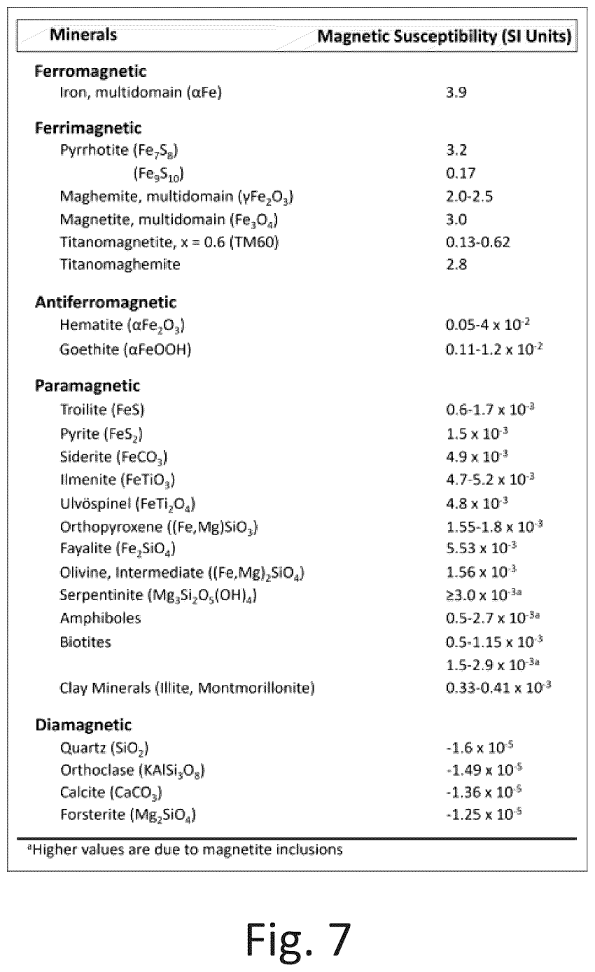

Magnetic susceptibility is a quantitative measurement of the extent to which a material may be magnetized in relation to an applied magnetic field. In other words, magnetic susceptibility indicates whether a material is attracted to or repelled by a magnetic field and the extent of this interaction. Five classifications of magnetic susceptibility exist, namely ferromagnetic, antiferromagnetic, ferrimagnetic, paramagnetic, and diamagnetic. Materials classified as ferromagnetic, antiferromagnetic, ferrimagnetic, and paramagnetic have positive magnetic susceptibilities and become magnetized in the direction of an applied field, while minerals classified as diamagnetic have a negative magnetic susceptibility and develop a magnetic moment opposite that of the applied field; higher absolute values suggest a stronger magnetic response in either direction (FIG. 7).

Ferromagnetic materials, such as native iron, have aligned magnetic domains that are parallel to the applied magnetic field, resulting in strong magnetic susceptibilities. Antiferromagnetism occurs when equivalent magnetic moments are aligned antiparallel to the magnetic field, causing the magnetism to disappear. Antiferromagnetic materials such as hematite exhibit weak, positive magnetic susceptibility when the magnetic moments are canted (i.e., tilted) away from perfect alignment, creating a weak magnetization perpendicular to the antiferromagnetism axis. The magnetic susceptibility of antiferromagnetic materials increases with temperature up to a mineral-specific value (i.e., Néel temperature) at which the material behaves like a paramagnetic material. Paramagnetism is caused by the spins of electrons that are not offset by opposing spins of other electrons in an electron shell, and these spinning electrons produce a magnetic dipole. In the absence of an external field, the net magnetic moment is zero. The magnetic susceptibility of paramagnetic materials also decreases with temperature. Strong carriers of paramagnetism (e.g., the ions Fe²⁺, Fe³⁺, and Mn²⁺) cause rock forming minerals such as orthopyroxenes (e.g., ferrosilite), clinopyroxenes (e.g., hedenbergite, augite), amphiboles, olivine, Fe- or Mn-containing serpentine minerals (e.g., either as polymorphs such as greenalite or replacements for Mg in lizardite or antigorite), and Fe-bearing metamorphic minerals such as chlorite, prehnite, and pumpellyite to be paramagnetic. Ferrimagnetic materials (e.g., magnetite) are the most common source of magnetism in geologic materials and occur when magnetic moments of the material are aligned antiparallel to the magnetic field in a similar manner as antiferromagnetic materials, but the magnetic moments are not equivalent in magnitude, creating a net magnetic moment (but relatively lower than ferromagnetic materials).

Both ferromagnetic and ferrimagnetic materials may also exhibit remnant magnetization, where the materials exhibit magnetic properties once an external field is removed. For example, geologic materials may acquire natural remnant magnetization during their formation due to the Earth's magnetic field and this property is often the basis of paleomagnetic studies. By comparison, paramagnetic materials generally do not exhibit remnant magnetization. Above elevated temperatures known as the Curie Temperatures specific to each material (e.g., ˜770° C. for native iron and ˜560° C. for magnetite), thermal energy is sufficient to maintain a random alignment of magnetic moments allowing ferromagnetic and ferrimagnetic materials to take on properties of paramagnetic materials without the associated strong magnetic susceptibilities or remnant magnetism.

In general, the relative magnetic susceptibilities of these materials may be summarized as ferromagnetic>ferrimagnetic>antiferromagnetic and paramagnetic>diamagnetic. More specifically: 1) ferromagnetic materials (e.g., metallic iron, nickel, cobalt), have the highest magnetic susceptibilities; 2) ferrimagnetic materials (e.g., magnetite, maghemite, pyrrhotite) display high magnetic susceptibilities, but lower than ferromagnetic minerals; and 3) antiferromagnetic (e.g., hematite) and paramagnetic (e.g., orthopyroxenes (ferrosilite), clinopyroxenes (hedenbergite, augite), amphiboles, olivine, Fe- or Mn-containing serpentine minerals (e.g., either as polymorphs such as greenalite or replacements for Mg in lizardite or antigorite), and Fe-bearing metamorphic minerals such as chlorite, prehnite, and pumpellyite; FIG. 4, FIG. 5) materials have relatively weak magnetic susceptibilities with magnetic susceptibility increasing in antiferromagnetic materials and decreasing in paramagnetic materials with increasing temperature. By comparison, diamagnetic minerals such as quartz, feldspars, halite, and calcite exhibit the lowest magnetic susceptibility.

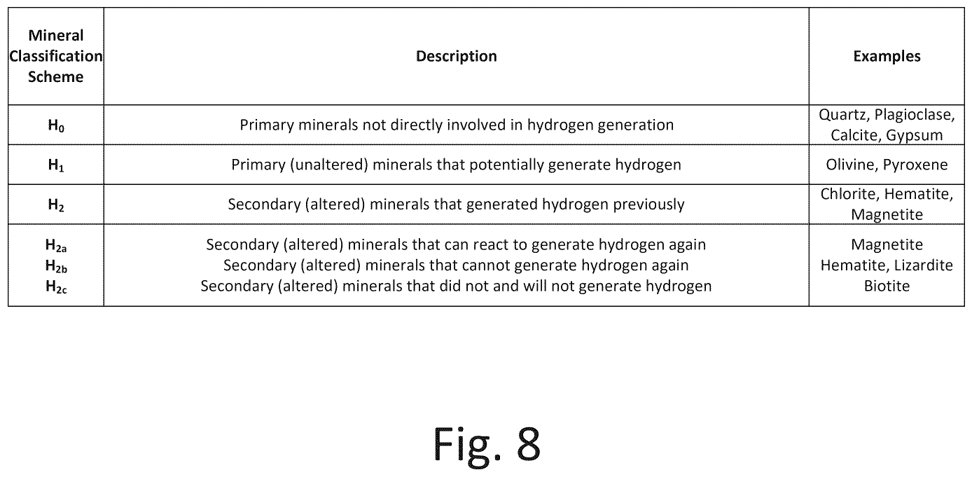

The minerals belonging to the classes of magnetic susceptibility described above are commonly found in hydrogen source rocks (FIG. 8). Ferrimagnetic minerals such as magnetite and maghemite have very high susceptibility values (˜3 SI units and ˜2-2.5 SI units, respectively), while antiferromagnetic minerals such as hematite (5×10⁻⁴-0.04 SI units), goethite (1.1×10⁻³-0.012 SI units), and ilmenite (4.5-5.2×10⁻³ SI units; ilmenite may be ferrimagnetic, antiferromagnetic, or paramagnetic depending on composition) exhibit lower magnetic susceptibility. Paramagnetic minerals, including (non-forsterite) olivine (1.56-5.53 ×10-3 SI units magnetic susceptibility), (non-enstatite) pyroxene (1.55-1.88×10⁻³ SI units magnetic susceptibility), clinopyroxenes (e.g., diopside, augite, and aegirine; 1×10⁴-4.4×10⁻² SI units depending on highly variable composition), biotite (0.5-2.9×10⁻³ SI units), amphiboles (0.5-2.7×10⁻³ SI units), and garnets (approximately 2×10⁻⁴-4.5×10⁻³ SI units for non-colorless varieties) have positive but relatively low magnetic susceptibility, making them only weakly magnetic. Diamagnetic minerals such as quartz (−1.6×10⁻⁵ SI units) and calcite (−1.36×10⁻⁵ SI units) have negative magnetic susceptibility, which means these materials repel an applied magnetic field, and have very low absolute magnetic susceptibility.

Due to the variations in the magnetic susceptibilities of minerals, variations in field-scale magnetic susceptibility of geologic materials can also result from differences related to specific rock properties. For example, changing mineral composition, variable lithological layering, changes in the oxidation state of given elements (Fe²⁺ versus Fe³⁺), chemical weathering processes, oxidation during mineral hydration, hydrothermal alteration, or metamorphic alteration processes can each cause appreciable differences in a rock's magnetic susceptibility. Generally, minerals that display the highest magnetic susceptibility include minerals such as ferrimagnetic iron-oxides (e.g., magnetite, maghemite) or iron-sulfides (e.g., pyrrhotite) (FIG. 7). Therefore, and by way of example, rocks that contain these minerals (e.g., serpentinite or serpentinized ultramafic and mafic rocks, banded iron formations) generally will have higher magnetic susceptibility than relatively iron-poor rocks (e.g., silica-rich igneous rocks like granites or phonolites, most sedimentary rocks). Further, alteration of low magnetic susceptibility, paramagnetic minerals (e.g., olivine, pyroxenes) produces ferromagnetic mineral phases (e.g., magnetite), increasing the overall magnetic susceptibility of the rock.

The proportions of ferrimagnetic magnetite compared to antiferromagnetic hematite may be of particular significance in understanding geologic hydrogen systems because these are important end products of many reactions involving iron-rich minerals that may contain iron in the Fe²⁺ or Fe³⁺ valance state. For example, hematite has significantly lower magnetic susceptibility (5×10⁻⁴ to 0.04 SI units) than magnetite (˜3 SI units) (FIG. 7). The oxidation of Fe²⁺ to Fe³⁺ reduces water, releases molecular hydrogen, and produces either magnetite in the absence of oxygen or the more stable mineral hematite if free oxygen is available. In contrast, alteration minerals that will or may incorporate ferrous iron without a change in oxidation state may have only small effects on magnetic susceptibility with respect to the original minerals (e.g., chlorite generated from alteration of olivine). Thus, utilizing magnetic susceptibility for the purposes of geologic hydrogen exploration requires a detailed understanding of the processes of oxidation, alteration, metamorphism, or other chemical reactions that may take place between geologic materials and their associated effects on magnetic susceptibility.

### Classification of Hydrogen Related Mineral Phases

For the purpose of hydrogen source rock evaluation, mineralogy can be classified into three general categories: 1) minerals that can be altered by the reactions described above and below to generate geological hydrogen, 2) minerals indicative of past generation of hydrogen in the system, and 3) minerals that are uninvolved in hydrogen generating reactions. The classification scheme developed for hydrogen-related mineral phases is discussed below and summarized in FIG. 8.

The primary minerals contained in hydrogen source rocks that are involved in hydrogen generation encompass a broad range of groups. The commonly known Fe2+-bearing silicate minerals capable of generating hydrogen include olivine (i.e., forsterite-fayalite: Mg—Fe solid solution series), orthopyroxene (i.e., enstatite-ferrosilite-pigeonite: Ca—Mg—Fe solid solution series), and clinopyroxene (i.e., diopside-hedenbergite-augite: Ca—Mg—Fe solid solution series). Although silicates such as olivine, orthopyroxene, and clinopyroxene are the most common sources for natural hydrogen formation, other mineral phases such as oxides (e.g., magnetite, ilmenite), spinels, amphiboles, garnets, inosilicates, sorosilicates, or others can also contribute to hydrogen generation.

Primary minerals can be further specified to determine the proportions of mineral types that generate hydrogen, sequester carbon, or sequester sulfur. One example is the mineral olivine (X₂SiO₄) where the X cation is a solid solution series between Mg and Fe, and where the molar abundance of Mg can react with CO₂ to form MgCO₃ while the molar abundance of Fe can react with H₂O to form Fe₃O₄ and release H₂ gas or with H₂S to form FeS (or other sulfides) and release H₂ gas, each in the presence of a reaction-appropriate fluid composition. In summary, the iron endmember can produce hydrogen, oxidize iron, and mineralize sulfur to make sulfides, while the magnesium and calcium endmembers can mineralize carbon to make carbonates during serpentinization processes. Generally, primary hydrogen minerals have magnetic susceptibilities that are elevated compared to minerals unrelated to hydrogen generation (e.g., quartz, plagioclase, calcite) but lower than secondary hydrogen minerals (e.g., magnetite, hematite).

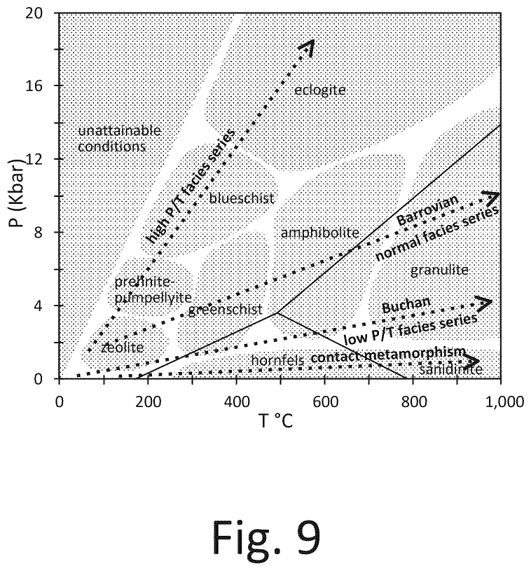

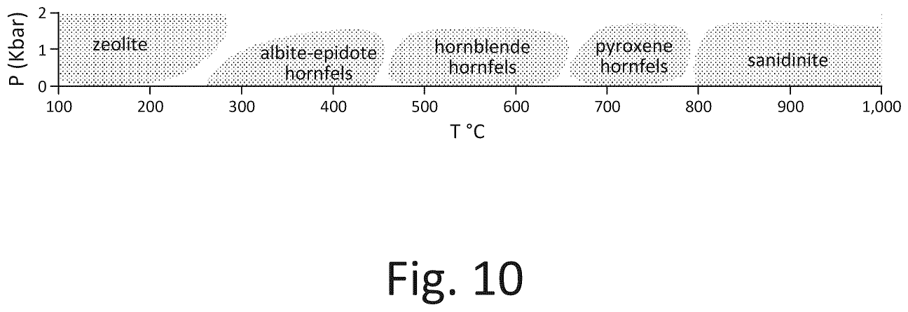

Metamorphism of the mineral phases containing high abundances of Fe²⁺ (e.g., minerals contained in mafic rocks) alters the primary minerals into various phases of secondary minerals whose formation is dependent upon the pressure, temperature, and fluid conditions associated with the metamorphic grade and pathways that the mafic rock experiences (FIG. 9, FIG. 10). Some secondary alteration mineral products may still retain some degree of Fe²⁺ that can generate additional hydrogen, including antigorite, chrysotile, cronstedtite, brucite, and lizardite, oxides (e.g., ilmenite, and magnetite), and others such as clays with varying degrees of Fe²⁺ (e.g., chlorite, smectite, vermiculite), carbonates (siderite), and spinels (e.g., magnetite, chromite, ulvöspinel, hercynite, magnesioferrite) and some low temperature and pressure metamorphic facies (e.g., zeolites, prehnite, pumpellyite). Some of these minerals are a solid solution of magnesium and iron (e.g., olivine is often made up of 85% forsterite (magnesium endmember) and 15% fayalite (iron endmember)). For all these minerals, the hydrogen generation potential will vary based on iron content, iron oxidation state, and that mineral's degree of reactivity.

Other secondary minerals represent the end product of completed alteration reactions that previously generated hydrogen (i.e., minerals containing Fe³⁺) and include ultramafic alteration products with varying degrees of Fe³⁺ (e.g., antigorite, chrysotile, cronstedtite, brucite, and lizardite), iron oxides (e.g., goethite, hematite, maghemite, magnetite), clays with varying degrees of Fe³⁺ (e.g., celadonite/glauconite, chlorite, kaolinite, smectite), and metamorphic facies with varying degrees of Fe³⁺ (e.g., epidote, garnets, prehnite, pumpellyite). Some of these minerals are associated directly with named metamorphic facies (FIG. 9). For all these minerals, past generation of hydrogen will again depend on iron content and iron oxidation state of the source material. Generally, secondary hydrogen minerals have magnetic susceptibilities that are elevated compared to primary hydrogen minerals (e.g., olivine, pyroxene) and significantly elevated compared to minerals unrelated to hydrogen generation (e.g., quartz, plagioclase, calcite).

Methods and systems for hydrogen source rock evaluation are developed [Eymold et al., 2024] for the purposes of understanding hydrogen systems, evaluating the quality of geologic hydrogen source rocks, and the potential for hydrogen to be generated by stimulated processes. Those improved estimates of source rock mineralogy and degree of alteration can be correlated with laboratory measurements of magnetic susceptibility and ground-based, sea-based, or airborne geophysical surveys or models (e.g., 2D/3D seismic, tomography, gravimetry) to extrapolate zones of interest and define target intervals and depths as part of geologic hydrogen exploration.

The terms above are written with respect to hydrogen generation but may also be considered for carbon mineralization and sulfur mineralization, as described in other disclosures. For example, stoichiometrically balanced equations that describe the reactions involving mineral phases that sequester carbon or sulfur during their formation (e.g., calcite, dolomite, pyrite) can be used to determine suitability for sequestration targets on a mole/unit mass of rock basis, analogous to evaluating hydrogen source rock potential. Mineral assemblages would also have to be categorized based on their role in mineralizing carbon or sulfur during their formation or providing potential pathways to sequester additional carbon or sulfur through reactions (e.g., carbonation, pyritization).

### Geologic Processes Related to Magnetic Susceptibility

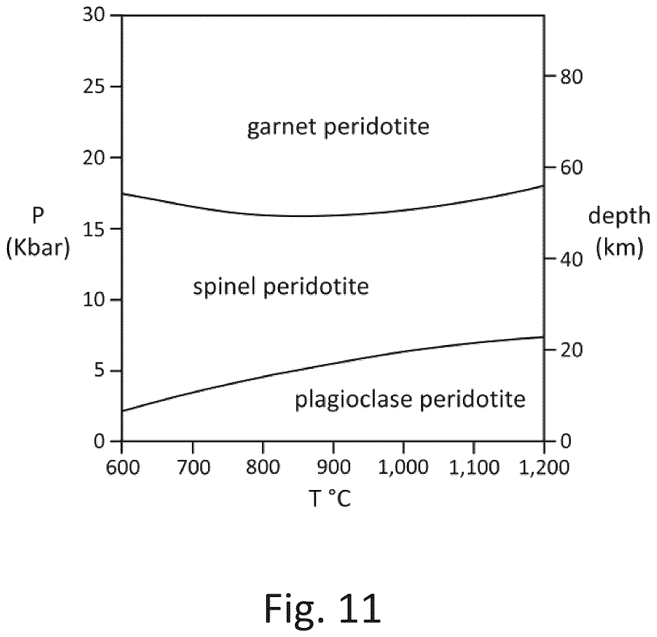

Variations in magnetic susceptibility are specifically observable in igneous rocks (FIG. 1) and mineral phases derived from the alteration of minerals in igneous rocks by chemical weathering, hydration, hydrothermal alteration, or metamorphism. For example, ultramafic magma crystallizing in different temperature and pressure regimes may lead to different mineral assemblages and thus different magnetic susceptibilities (FIG. 11), or mixing of magma from different sources (i.e., combining magmas of different compositions) may create hybrid magmas with mixed magnetic susceptibilities, variable magnetic susceptibilities, or in some cases divergent magnetic susceptibilities due to the extent of mixing throughout an igneous body.

Beyond these examples of differences in magnetic susceptibilities related to chemical heterogeneity in igneous rock composition, processes during magmatic emplacement may also result in variations in magnetic susceptibility. One illustrative example of changes in magnetic susceptibilities is related to the processes of fractional crystallization during the cooling of magma in magma chambers in which solidifying crystals become denser than the surrounding melts and are physically separated from cooling magma. In this scenario, denser mineral phases such as olivine and pyroxene, which are rich in compatible elements such as magnesium and iron, are systematically (and often predictably) deposited near the bottom of magma chambers or layered mafic intrusions, while the remaining melt becomes enriched in more incompatible elements, including silicon, calcium, and potassium. These processes lead to a progressive enrichment in quartz, plagioclase, and alkali feldspars near the top of magma chambers or layered mafic intrusions. As a result, even individual magma chambers can display complex and variable rock properties such as magnetic susceptibility, density, and mineralogy.

Additional magmatic processes such as partial melting of a magma source or crustal assimilation of continental crust or country rock produce igneous products with distinct compositions and rock properties. Metasomatism, the chemical alteration by hydrothermal fluids, also changes mineral composition in localized features and can create great heterogeneity in magnetic susceptibility. These processes, individually or combined, all lead to the complex chemical and mineral heterogeneity within igneous rocks.

Additional processes exist that may cause differences in magnetic susceptibility related to the emplacement of extrusive igneous rocks (i.e., magma cooled at or near the surface) in particular. Partial melting or assimilation of country rock into the feeder magma, mineralogical or chemical compositional variations, grain size, and the petrology/crystallography (e.g., glass versus crystals versus ash) of igneous materials, especially extrusive volcanics, can also produce significant differences in the resulting magnetic susceptibility. Materials that may include pyroclastics such as ash or tuff, cinder, or lahar flows (i.e., a downslope flowing slurry that can include mud, water, and ash), or lava flows can blanket the Earth's surface sequentially, resulting in layers of these materials that may exhibit variations in magnetic susceptibility due to the amount of iron-bearing mineral phases contained in each phase or by mixing/dilution with non-magnetic material. For example, a bimodal volcanic system where both mafic and felsic lavas are ejected from a single volcanic center may have layers of high magnetic susceptibility (mafic rock) and low magnetic susceptibility (felsic rock) material. Ash falls or tuffs also generally result from explosive eruptions of felsic (high silica, low iron and magnesium) materials that may be ejected and transported over great distances and cover large spatial areas. The ash falls and tuffs both tend to be characterized by relatively low magnetic susceptibilities as compared to mafic lavas.

Variable weathering processes at or near the surface may alter the mineralogy and therefore magnetic susceptibility of the emplaced volcanic materials. For example, weathering, hydration, and oxidation processes that alter extrusive igneous rocks occur near the surface and produce differences in magnetic susceptibility during or after the solidification or crystallization of the lava (or magma in the case of shallow emplacement). The elevated levels of oxygen at or very near the surface (e.g., air reacting with extrusive bodies, air reacting with bodies brought to the surface by erosion or uplift, or in shallow (e.g., <100 meters) oxygenated ground or surface waters) can lead to extensive alteration of iron-containing minerals sourced from igneous rocks. In shallow environments, these chemical weathering processes often lead to iron oxidation and the formation of hematite or iron clay minerals (e.g., goethite). Alternatively, hematite can form in relatively oxygen free environments from the alteration of primary mafic minerals at high water-rock ratios, alteration of magnetite, and following the oxidation of secondary iron-rich minerals that contain abundant Fe²⁺ (e.g., brucite, chlorite, prehnite, pumpellyite, serpentine group minerals) during later stages of mafic metamorphism in instances where alternative electron donors have already been exhausted.

### Laboratory and Wireline Based Methods to Measure Magnetic Susceptibility

Existing systems and methods of measuring magnetic susceptibility and embodiments of the systems and methods relating to the unique utilization of these tools are hereafter described. Examples of instrumentation types that can be used to quantify magnetic susceptibility may include using a Vibrating Sample Magnetometer (VSM), Superconducting Quantum Interference Device (SQUID) magnetometer, Magnetic Property Measurement System (MPMS), AC susceptometer, Kappabridge, Faraday Balance, Gouy Balance, alternating gradient magnetometer, a cable-based downhole version of any of these tools used to measure magnetic susceptibility in situ in a well or borehole, or other instrumentation meant for quantifying magnetic susceptibility in geologic materials, or combinations thereof. Magnetic susceptibility may also be estimated through computer-based modelling of regional magnetic data (e.g., Werner deconvolution and inverse modelling).

A Vibrating Sample Magnetometer (VSM) utilizes a uniform magnetic field in which a sample is mechanically vibrated to detect a magnetic response through induced voltage in nearby pickup coils. Prior art discloses the characterization of the magnetic properties of materials and analysis of rock samples to understand the Earth's magnetic properties. The VSM uses units of magnetic field strength in tesla (T) and magnetic moment in units of ampere-meter squared (A·m²) or emu (electromagnetic units). The magnetic moment can also be expressed in units of Bohr magneton (μB) or nuclear magneton (μN) for atomic or molecular systems.

SQUID (Superconducting Quantum Interference Device) magnetometers measure magnetic susceptibility with high sensitivity by exploiting superconducting quantum interference. Operating within cryogenic environments, they detect infinitesimal changes in magnetic flux induced by an external field applied to a sample. The field can be static (DC) or dynamic (AC) and induced changes can be converted into voltage signals, enabling precise determination of magnetic susceptibility. Prior art of this instrumentation includes disclosures in geophysics and geology. In geophysics, it maps magnetic anomalies for mineral deposit exploration and subsurface formations for oil/gas exploration. In geologic applications, it is used to gain insight into formation history and composition. It has not been used for hydrogen exploration. SQUID magnetometers measure magnetic fields in tesla (T) or gauss (G), with 1 tesla equaling 10,000 gauss. These instruments measure magnetic moments in ampere-meter squared (A·m²) or erg per gauss (erg/G), and magnetic flux in weber (Wb) or maxwell (Mx). Magnetic susceptibility is dimensionless, and magnetization is measured in ampere per meter (A/m) or emu per cubic centimeter (emu/cm³), with 1000 A/m equaling 1 emu/cm³.

The Magnetic Property Measurement System (MPMS) is used for the precise measurement of magnetic properties in a wide range of materials. Operating based on the principles of magnetometry, the MPMS can characterize various magnetic phenomena, including magnetization, magnetic susceptibility, and magnetic hysteresis. Prior art disclosed explores the utilization of this system to study the magnetic behavior of materials under different conditions, such as temperature and applied magnetic field strength. Other applications of prior art leverage the high sensitivity and accuracy of the MPMS to facilitate detailed investigations into the magnetic properties of materials in fields such as geology where it is used to analyze ore deposits, conduct paleoclimate sediment analysis, and identify magnetic phases, but it has not been used for hydrogen exploration. Its non-destructive nature and ability to perform measurements over a broad range of temperatures and magnetic fields make the MPMS an invaluable tool for understanding the fundamental magnetic properties of materials and their practical applications in technology and industry.

AC susceptometers are utilized to measure the alternating magnetic susceptibility of materials as a function of frequency and temperature. Operating based on the principles of magnetometry, the AC susceptometer generates an alternating magnetic field that oscillates at a specific frequency, inducing a magnetic response in the sample under study. By detecting the changes in magnetic flux or magnetization induced by the alternating field, the instrument can record the amplitude and phase of the magnetic response across a range of frequencies. This allows researchers to extract valuable information about the magnetic properties of the sample, including its susceptibility, relaxation behavior, and magnetic dynamics. Such methods have not been used to explore for geologic hydrogen, identify geologic hydrogen source rocks, or distinguish regions or degrees of hydrogen generation. AC susceptometers measure magnetic susceptibility, typically reported as a dimensionless quantity (SI). The alternating magnetic field strength is measured in units of tesla (T) or gauss (G). The resulting magnetization of the sample is measured in units of amperes per meter (A/m) or electromagnetic units per cubic centimeter (emu/cm³).

A Kappabridge is utilized to measure the magnetic susceptibility of rocks, sediments, and other materials. It operates by placing a sample on an oscillating mechanism with a 3D rotator. The magnetic susceptibility of the sample influences its response to the magnetic field, which is detected by a sensitive coil arrangement within the device. The Kappabridge measures both the in-phase and out-of-phase components of the sample's magnetic response, providing information on both the magnetic susceptibility and potential viscous effects. These measurements are processed and analyzed to determine the magnetic properties of the material, which are crucial for geological and environmental studies. The Kappabridge has not been used to explore for geologic hydrogen, identify geologic hydrogen source rocks, or distinguish regions or degrees of hydrogen generation. A Kappabridge measures magnetic susceptibility, typically reported in dimensionless SI or CGS units. However, for practical purposes, these measurements are often expressed in terms of volume magnetic susceptibility (κ), which can be reported in units of 10⁻⁶ (SI) or 10⁻⁶ (CGS). The instrument applies an oscillating magnetic field, usually measured in units of microteslas (μT), to the sample.

A Faraday balance measures the magnetic susceptibility of a sample based on the force it experiences in a non-uniform magnetic field. The core principle involves placing the sample in a magnetic field gradient generated by a pair of strong magnets. The sample experiences a force proportional to its magnetic susceptibility and the gradient of the magnetic field. This force causes a measurable deflection of a sensitive balance beam or torsion wire on which the sample is mounted. The deflection is detected and measured, often using optical or electronic methods, and is directly related to the magnetic susceptibility of the sample. By precisely measuring this force, the Faraday balance provides accurate information about the magnetic properties of the material. This technique is particularly useful for studying the magnetic susceptibility of liquids and small solid samples in research fields such as materials science, chemistry, and physics. The Faraday balance measures the magnetic susceptibility of a sample, typically expressed in dimensionless SI units for specific susceptibility (χ). The magnetic field strength applied to the sample is measured in units of tesla (T) or gauss (G). The force experienced by the sample in the magnetic field gradient is measured in newtons (N).

A Gouy balance measures the magnetic susceptibility of a sample by analyzing the change in its apparent weight when placed in a magnetic field. The setup involves suspending a sample in a uniform magnetic field created between the poles of an electromagnet. When the sample, often in the form of a cylindrical tube filled with the material, is introduced into the magnetic field, it experiences a force due to its magnetic susceptibility. This force causes a slight change in the apparent weight of the sample, which is measured with a highly sensitive balance. The change in weight is proportional to the magnetic susceptibility of the sample. By carefully measuring the difference in weight with and without the application of the magnetic field, magnetic susceptibility can be calculated. This technique is particularly useful for measuring the magnetic properties of liquids, powders, and other small samples in fields such as chemistry and materials science. The Gouy balance measures the magnetic susceptibility of a sample, typically reported in dimensionless SI units. The magnetic field strength applied to the sample is measured in units of tesla (T) or gauss (G). The change in the apparent weight of the sample in the magnetic field is measured in units of newtons (N), reflecting the force exerted on the sample due to its magnetic susceptibility.

An alternating gradient magnetometer is a specialized instrument used for measuring the magnetic properties of materials, particularly those with low-field magnetic susceptibility. The alternating gradient magnetometer operates by subjecting the sample to an alternating magnetic field produced by a pair of gradient coils. These coils generate a magnetic field gradient across the sample, which induces a response proportional to its magnetic susceptibility. By varying the frequency and amplitude of the alternating field, the instrument can precisely measure the sample's susceptibility over a wide range of frequencies and temperatures. The induced response is detected and analyzed using sensitive detectors, providing valuable insights into the magnetic behavior of materials. The alternating gradient magnetometer has not been used for the exploration of geologic hydrogen. An alternating gradient magnetometer typically measures magnetic susceptibility, which is reported in dimensionless SI units. The alternating magnetic field strength applied to the sample is measured in units of tesla (T) or gauss (G).

Instruments lowered by cable into a well or borehole (i.e., attached to a wireline tool, wireline logging, or “logging”) are frequently used to make measurements of rock, sediment, and fluid characteristics (e.g., density, porosity, natural radioactivity, acoustic response, and fluid resistivity) across the depth of a well or borehole in the exploration and extraction of oil, hydrocarbon gases, helium, carbon dioxide, as well as in the geothermal and mining industries. This may take place immediately upon the completion of the drilling of a well or borehole, or in intermediate stages when the drill string and bit are temporarily lifted up and out of the well or borehole and replaced by tools. Similar wireline tools exist for measuring magnetic susceptibility in a borehole or well but have not previously been conceived of as relevant to explore for geologic hydrogen, identify geologic hydrogen source rocks, or distinguish regions or degrees of hydrogen generation. Prior art of magnetic susceptibility wireline tools has used these tools (e.g., a magnetic susceptibility sonde) to measure magnetic susceptibility across sediments and rocks, often for the purposes of understanding the iron oxidation states of the geologic material along chemical or biologic reaction fronts or to identify zones of hydrothermal alteration related to ore mining deposits, but not for the purpose of exploring for natural hydrogen, evaluating the presence, quality, or maturity of hydrogen source rock (i.e., the extent to which a rock has advanced along the pathway of hydrogen generation), or the identification of potential locations for engineered stimulated hydrogen generation, carbon mineralization, or sulfur mineralization.

### Combining Magnetic Susceptibility Measurements with Other Geophysical Data to Improve Geologic Hydrogen Exploration

The alteration or metamorphism of mafic or ultramafic rocks has significant potential to change the consortium of alteration minerals observed and thereby the magnetic properties and densities of the resulting rocks and minerals to influence the hydrogen generation efficiency per unit rock based on the abundance and speciation of Fe²⁺. A variety of parameters can contribute to mineral alteration assemblages produced from the metamorphism of mafic rocks, influencing both a rock's hydrogen generation capability and its measured magnetic properties. Referring to FIGS. 9 and 10, several examples of rock properties that influence hydrogen generation volumes and magnetic susceptibility include the initial composition of minerals present in source rocks, the temperature conditions of metamorphism, the pressure conditions of metamorphism (i.e., often called the metamorphic grade), the temporal conditions and kinetic rates of metamorphism, and the anticipated volumes and compositions of fluids that are available to react with prospective natural hydrogen source rocks. Each of these parameters may lead to measurable and predictable changes in magnetic susceptibility, density, and other rock properties, which can be evaluated on macroscopic levels by ground-based, sea-based, or airborne gravity and magnetic surveys. As a result, each of these parameters should be suitably constrained to evaluate prospectivity for natural hydrogen accumulation and/or direct evidence of hydrogen generation and be suitably calibrated in order to properly map depths, elevations, dips, and other parameters of mafic rocks and their alteration products.

Thus, the methods described herein enable the ability to distinguish: 1) mafic rocks from ultramafic rocks; 2) mafic/ultramafic rocks from altered or metamorphosed mafic/ultramafic rocks; 3) mafic/ultramafic rocks from other non-mafic igneous or metamorphic rocks; 4) mafic/ultramafic rocks from sediment or sedimentary rocks; 5) serpentinized rocks from other lower quality mafic/ultramafic alteration products; 6) pyrite from mafic/ultramafic rocks; 7) pyrite from alteration products of mafic/ultramafic rocks; and 8) sedimentary rocks rich in mafic minerals or iron (e.g., basaltic sandstone or banded iron formation) from iron-poor sedimentary rocks or sediment. The ability to resolve or distinguish each of these conditions improves upon current methods and models of geologic hydrogen exploration.

Interpreting aeromagnetic data in the context of well-characterized, high-resolution magnetic susceptibility data for key mineral phases enables the development of accurate geomodels of key hydrogen system components (e.g., source rocks) and robust maps of mafic surfaces, altered mafic surfaces, and overlying sediments. The parameters and associated algorithms for interpretation disclosed herein enable natural hydrogen exploration workflows designed to prospect for natural hydrogen accumulations and to distinguish target areas for engineered hydrogen production or stimulation, carbon mineralization, or sulfur mineralization in the subsurface.

Further, when used in combination with mineralogical or lithological data, magnetic susceptibility measurements of materials collected from a well or borehole or collected from the surface may be used to generate more accurate regional geological interpretations. Tying the high resolution, localized magnetic susceptibility data to data generated from regional geophysical surveys helps identify hydrogen source rocks or zones favorable for hydrogen accumulation across a region of interest. The utilization of magnetic susceptibility may be further enhanced with mineral observations, density observations, or gravity measurements.

### Acquisition of High-resolution Magnetic Susceptibility Data

Mineralogical or lithological information obtained from drill cuttings, rotary sidewall cores, or whole cores can be described and/or characterized by optical mineralogy or measured by various analytical techniques, such as x-ray diffraction (XRD), x-ray fluorescence (XRF) and scanning electron microscopy (SEM). The direct measurement of these components at discrete intervals in combination with magnetic susceptibility can be used to identify and differentiate rock types and mineral phases that result from the alteration of mafic and ultramafic rocks, to differentiate mafic and ultramafic rocks from other rocks that are not capable of serving as natural hydrogen source rocks, and to discretely evaluate the presence and relationship of these rocks along the depths of a well or borehole.

In an embodiment, magnetic susceptibility may also be used in combination with normative mineralogy to determine the extent to which hydrogen may have already been generated from a natural hydrogen source rock, referred to as “past hydrogen generation,” and the extent to which hydrogen may still be generated with further reactions, referred to as “future hydrogen generation,” can be quantified. Normative mineralogy involves the calculation of an idealized original igneous rock mineralogy based on the bulk elemental composition (e.g., from XRF), that is then compared to current mineralogy (e.g., from XRD) and known mineral alteration pathways of mafic rocks. In determining past and future generation, the relative quality of a hydrogen source rock may be determined and associated with specific magnetic susceptibility ranges within a given source rock body, which can then be used as exploration targets.

The identification of prospective hydrogen source rock within a well or borehole also enables the development of a geologic hydrogen system. One embodiment relates to the use of these high-resolution data obtained from a well or borehole (i.e., drill cuttings, whole cores, rotary sidewall cores, or wireline data) to develop and/or calibrate geophysical models that are used to either contextualize ground-based, sea-based, or airborne magnetic data or to extrapolate specific magnetic characteristics (especially when combined with mineral models or gravity measurements) to broader geologic regions of interest for ongoing exploration. Once the magnetic susceptibility character of a validated geologic hydrogen source rock can be determined, regional-scale ground-based, sea-based, or airborne magnetic data can be used to search for the signal of interest to map the prospective zones of natural hydrogen generation.

The ability to robustly map natural hydrogen source rock and to evaluate the relative quality of that source rock across a region also enables identification and focused mapping and evaluation of other components of the natural hydrogen system, such as but not limited to prospective migration pathways and mechanisms, reservoir, trap, and seal components of natural hydrogen systems. Careful evaluation and mapping of these components allows for developing prospects or areas that can accumulate natural hydrogen. Other data types, such as hydrogen source rock evaluation, 2D, 3D, and tomographic seismic imaging, geologic mapping, as well as drilling data, such as wireline logging, hydrogen shows, successful drill stem tests, flow tests, production tests, or other tests evaluating the fluid from specific subsurface intervals may also be incorporated into the processes described herein.

In an embodiment, well or borehole magnetic susceptibility measurements (e.g., drill cuttings, core, or wireline data) are used to link well or borehole data with ground-based, sea-based, or airborne magnetic surveys. Depth and magnetic susceptibility data from a well or borehole can be tied to magnetic field datasets for an outcome similar to what is achieved through the methods of using data from a vertical seismic profile (VSP), sonic, density, or another wireline logging tool of a drilled borehole to create a seismic well tie. A seismic well tie is a fundamental step in interpreting seismic reflection data. Seismic well ties provide a numeric match between acoustic responses to lithologies across the depth of the borehole and seismic reflection data, which is measured in time, thus providing a precise method of converting time data to depth data. Converting seismic data from time to depth is critical in the placement of exploration wells, generating a geoprognosis, picking target depths, mapping reservoir and seal formations, and quantifying reservoir or pay zone volumes. Similarly, in this embodiment, magnetic susceptibility data from geological material from a well or borehole (e.g., cuttings, core, wireline tool, or other) can be “tied” to regional magnetic field data.

In an embodiment, magnetic susceptibility may be used to identify discrete zones within hydrogen source rocks (e.g., mafic and ultramafic rocks) that serve as unconventional-style hydrogen reservoirs where hydrogen may have been generated at some point in the geologic past. This may include measurements from discrete intervals in a well or borehole, as well as measurements from rocks collected at the surface, which can be extrapolated to mappable hydrogen source rocks (e.g., mapped by ground-based or airborne gravity and magnetics data) at depth.

Once a rock has been identified as a potential hydrogen source rock, relative changes in magnetic susceptibility within the source rock bodies may reflect changes in mineralogy related either to differences in the original rock composition (e.g., by fractional crystallization, intrusive versus extrusive emplacement) or varying degrees or Pressure-Temperature-time (P-T-t) pathways of mineral alteration following emplacement (FIG. 6). For example, variations in rocks that were altered to serpentine group minerals and produced hydrogen as opposed to being altered by various other mafic metamorphism pathways that can produce hydrogen would exhibit increases in the intensity of their magnetic susceptibility due to increases in magnetite, as well as differences in other rock properties such as density/gravity, or mineralogy.

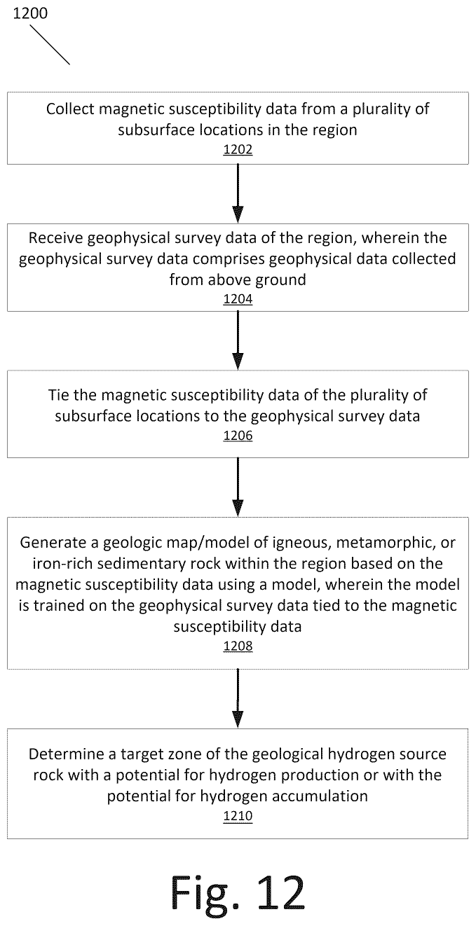

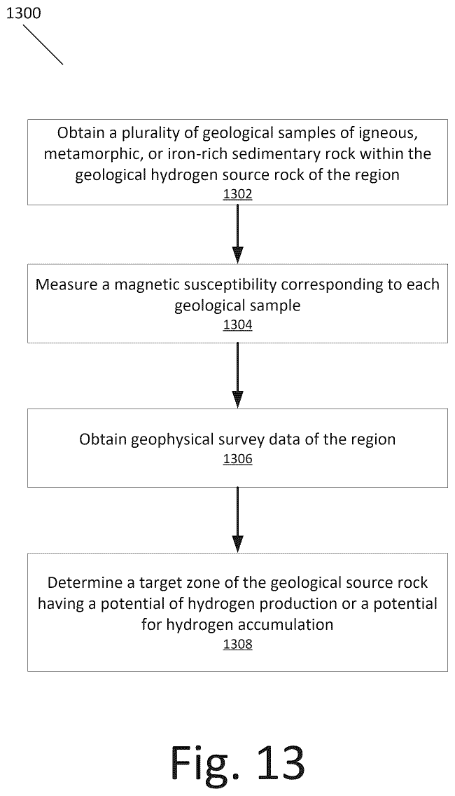

FIGS. 12-13 illustrate various methods for evaluating geological hydrogen source rock in a region. The following descriptions detail the steps of the methods. It is understood that the steps described for each method could be used in the others.

Referring to FIG. 12, a method 1200 for evaluating geological hydrogen source rock in a region is provided. In the first step 1202, magnetic susceptibility data is collected from a plurality of subsurface locations within the region. To collect the magnetic susceptibility data, a borehole may be drilled such that the plurality of subsurface locations is located within the borehole. The magnetic susceptibility may be measured for a geological sample taken at each subsurface location using a vibrating sample magnetometer, a superconducting quantum interference device magnetometer, a magnetic property measurement system, an AC susceptometer, a Kappabridge, a Faraday balance, a Gouy balance, an alternating gradient magnetometer, a wireline magnetic susceptibility tool, or another suitable device.

Further, in some embodiments, the subsurface locations span a vertical axis through the region, and the magnetic susceptibility data includes a magnetic anomaly at a subsurface location above, below, or within the igneous rock, metamorphic rock, or mafic mineral-or iron-rich sedimentary rock within the region. For example, the subsurface locations may include one or more locations in a sedimentary basin, such as an intra-rift sedimentary basin or a back-arc sedimentary basin, and one or more locations in the igneous or metamorphic rock within or below the sedimentary basin. The magnetic anomaly may correspond to a change in concentration or type of minerals relative to the surrounding area. In some embodiments, the magnetic anomaly may reflect, for example, a deviation from the magnetic susceptibility of nearby minerals within a range of at least about 3%. In other embodiments, the deviation may be much larger, and still other embodiments, the deviation may be smaller.

A geophysical survey data of the region including geophysical data collected from above ground is received in step 1204. The geophysical survey data may include airborne magnetic field measurements derived from airborne magnetic surveys or electromagnetic surveys, gravity data provided from airborne gravity or gravity gradiometers, airborne gravity and magnetic surveys, ground-based gravity and magnetic surveys, sea-based gravity and magnetic surveys, or electromagnetic surveys, and/or seismic data including one of two-dimensional or three-dimensional seismic reflection or refraction seismic data or seismic tomography data. As a result, the geophysical survey data may include one or more of mineralogical data, magnetic field data, magnetic field gradient data, gravitational data, gravitational gradient data, spatially resolved well data, wireline data, wireline instrumentation data, wireline density data, acoustic impedance data, seismic data, or electromagnetic data.

The magnetic susceptibility data is then tied to the geophysical survey data in step 1206, which is then used to generate a geological map or model of the igneous rock, metamorphic rock, or mafic mineral-or iron-rich sedimentary rock within the region in step 1208. The geological map or model is generated using a model that is trained on the geophysical survey data tied to the magnetic susceptibility data. The model may be, for example, a statistical model. As discussed herein, the model may be a Werner deconvolution inverse model or a Werner deconvolution forward model. In some embodiments, the method 1200 includes a further step of determining mineralogy of each geological sample. The mineralogy may be determined by x-ray diffraction, x-ray fluorescence, scanning electron microscopy, or scanning electron microscope-energy dispersive x-ray spectroscopy. The mineralogy may be normative or modal mineralogy.

Using the geological map or model, a target zone of the geological hydrogen source rock with a potential for hydrogen production or with the potential for hydrogen accumulation is identified or determined in step 1210. In some embodiments, the target zone is a target zone of potential hydrogen production corresponding to lithological zones of source rock capable of generating hydrogen. In other embodiments, the target zone is a target zone of potential hydrogen accumulation corresponding to lithological zones of source rock capable of serving as a reservoir for hydrogen accumulation. The lithological zones may correspond to source rock capable of providing hydrogen derivatives, carbon mineralization, or sulfur mineralization naturally or upon injection of one or more reactants.

FIG. 13 illustrates another method for evaluating geological hydrogen source rock in a region. In the first step 1302, a plurality of geological samples of igneous rock, metamorphic rock, or mafic mineral-or iron-rich sedimentary rock within the geological hydrogen source rock of the region are obtained. The magnetic susceptibility of each geological sample is measured in step 1304. Geophysical survey data of the region is obtained in step 1306, and, using the magnetic susceptibility of the plurality of geological samples and the geophysical survey data, a target zone of the geological hydrogen source rock with a potential for hydrogen production or with the potential for hydrogen accumulation is identified or determined in step 1308.

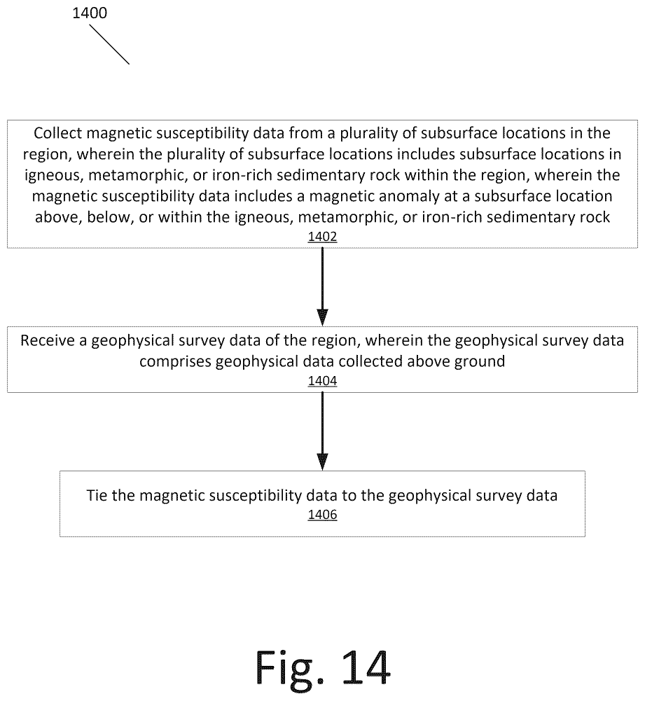

FIG. 14 illustrates a further method for evaluating geological hydrogen source rock in a region. In a first step 1402, magnetic susceptibility data is collected from a plurality of subsurface locations within the region, including subsurface locations in igneous rock, metamorphic rock, or mafic mineral-or iron-rich sedimentary rock within the region. The magnetic susceptibility data includes a magnetic anomaly at a subsurface location above, below, or within the igneous rock, metamorphic rock, or mafic mineral-or iron-rich sedimentary rock within the region.

In a second step 1404, geophysical survey data of the region collected above ground is obtained. The magnetic susceptibility data is tied to the geophysical survey data in step 1406.

In some embodiments, the intensity of magnetic susceptibility may be used to differentiate the quality of prospective hydrogen source rocks. Depending on the original rock composition or mineralogy and the pore fluid composition, pressure, temperatures, and kinetic rates of alteration reactions, various altered mineral phases (e.g., serpentine group vs. prehnite-pumpellyite vs. amphibole phases (see FIGS. 4-6)) can form, which may affect the efficiency of hydrogen generation from various source rocks and which display a range of diagnostic magnetic susceptibilities. Magnetic susceptibility can be used to evaluate those differences, identifying the differing capacities of various target lithologies to generate hydrogen, or quantify the proportion of hydrogen that can be generated in a given unit of rock. Therefore, an embodiment of this disclosure may include the use of magnetic susceptibility in combination with other tools (e.g., wireline density or elemental spectroscopy tools) or mineralogical or lithological information (e.g., drill cuttings description, XRD, XRF, SEM) to identify intervals within source rock bodies that represent evidence of past hydrogen generation, to provide input to algorithms for the quantity of hydrogen generation, or to identify zones of unaltered hydrogen source rocks (i.e., immature source rocks) or the proportion of unaltered rocks.

As an example, lithologic units within an ultramafic rock that contain serpentine—evidence that serpentinization or other hydrogen-generating alteration reactions has occurred and therefore hydrogen has been generated—exhibit increased magnetic susceptibility due to an increase in magnetite mineral phases. These interpretations may be further evident when integrating other data types, such as hydrogen in mud gas (i.e., gas extracted from drilling fluid during the drilling of a well or borehole), drill stem tests, flow tests, production tests, or other tests meant to evaluate fluids from specific subsurface intervals, with magnetic susceptibility measurements.

Multiple mechanisms may lead to the presence of hydrogen in specific formations or intervals, which may then manifest as mud gas during drilling or in drill stem tests, flow tests, production tests, or other tests meant to evaluate fluids from specific subsurface intervals. Once generated, gaseous hydrogen may remain dissolved in pore water until a saturation level is reached, at which point a gas phase will be created. Gaseous hydrogen can then migrate buoyantly upwards through water-saturated rock formations until reaching a formation that is of sufficiently low porosity and permeability to inhibit the further migration of hydrogen, acting as a seal. Generally, porous and permeable rock formations underneath low porosity and low permeability seals may serve as reservoirs where geologic hydrogen or other natural gases (e.g., helium, CO₂, H₂S, hydrocarbons) can accumulate. Recently generated hydrogen may mix with other gases present in pore spaces either within the source rock (e.g., CO₂), after migration out of source rock (e.g., during buoyant migration or at a zone of accumulation), or as a result of mixing with other natural gases that may be migrating through the hydrogen source rock (e.g., helium, CO₂, H₂S, hydrocarbons, steam). These other gases may also facilitate the migration of hydrogen as the hydrogen becomes part of a larger gas volume mixture at a greater pressure or with more buoyancy force.

Magnetic susceptibility used in combination with tools to identify porosity may identify intra-source rock reservoirs or zones of accumulation within source rocks that are sealed by other formations or low permeability units within the source rock. Tools to identify porosity, such as matrix porosity or fracture porosity, include density porosity, neutron porosity, far-field sonic, nuclear magnetic resonance (NMR), or Formation MicroImager (FMI) or similar wireline tools.

In an embodiment, magnetic susceptibility may be used to identify discrete zones within hydrogen source rocks (e.g., mafic and ultramafic rocks) that are relatively unaltered.

By way of example, intervals within hydrogen source rocks that have not undergone serpentinization or other hydrogen-generating reactions, oxidation, metamorphism, or other alteration processes may have relatively low magnetic susceptibility due to the presence of unaltered, weakly magnetic minerals that have a high capability to generate hydrogen or sequester carbon or sulfur through mineralization. Examples of such minerals include olivine and pyroxene, as shown in FIG. 3. Notably, unaltered hydrogen source rocks such as mafic and ultramafic rocks, which can be readily distinguished by their lower (relative to altered hydrogen source rock) magnetic susceptibility, are anticipated to display relatively low degrees of natural fracture generation and are therefore expected to have extremely low natural porosity and low permeability. Thus, another embodiment of this disclosure involves using measurements of magnetic susceptibility to identify zones that can behave as sealing lithologies for natural hydrogen accumulations within the source. Incorporation of borehole image logs or other logging data likely improves the capability of magnetic susceptibility to identify these units. Sealing lithologies in natural hydrogen source rocks can also be formed if carbon mineralization, clay formation, or other minerals fill natural fracture sets. Some relevant examples from natural hydrogen systems include carbon mineralization during serpentinization or clay formation following mafic/ultramafic rock metamorphism. In each of these cases, the relative changes in magnetic susceptibility in discrete lithologic units can be used to identify prospective sealing lithologies, especially in combination with other techniques (e.g., borehole image analyses). An embodiment of this approach is the ability to use high-resolution magnetic susceptibility data to identify prospective sealing lithologies. Other embodiments include integrating high-resolution magnetic susceptibility values (i.e., drill cuttings, whole cores, rotary sidewall cores, or wireline magnetic data) with standard wireline logs (e.g., bulk density, neutron, resistivity logs) more commonly used to evaluate rock properties and sealing potential.

### Magnetic Susceptibility to Detect Source Rock or Reacted Lithologies

In further embodiments, measurements of magnetic susceptibility from one or more wells or boreholes, either via drill cuttings, core, or wireline data, may be used to extrapolate identified hydrogen source rock, zones within hydrogen source rock that have evidence of hydrogen generation, or zones of hydrogen accumulation within one or more wells or boreholes across broader regions (laterally or obliquely away from the well or borehole) in a survey area for the purposes of identifying additional potential hydrogen source rock, zones within hydrogen source rock with potential hydrogen generation, or zones of potential hydrogen accumulation (e.g., reservoir) that may serve as future hydrogen exploration targets. This can be done by applying high-resolution magnetic susceptibility data to calibrate magnetic and other geophysical models or improve magnetic field interpretations. This extrapolation can be used alone or in combination with other well or borehole data (e.g., drill cuttings description, XRD, XRF, SEM, or hydrogen in mud gas, drill stem tests, flow tests, production tests, or other tests meant to test fluids from specific intervals in a well or borehole).

In this embodiment, the measured magnetic susceptibility across the depth of a single well or borehole or multiple wells or boreholes can be used to extrapolate across broader, regional 2-dimensional cross sections (i.e., a single survey line) or 3-dimensional volumes (i.e., portions or all of a survey area) of magnetic susceptibility estimated by geologic models. This can be accomplished, for example, by mapping horizons or bodies of equivalent or similar magnetic susceptibility values using subsurface geological interpretation software (e.g., Petrel®, IHS Kingdom Suite®, or others) in a similar fashion to mapping acoustic impedance contrast horizons on a 2-D seismic, 3-D seismic, or tomographic seismic section. In doing so, any mineralogical or lithological data, hydrogen gas data (e.g., presence of elevated hydrogen in mud gas, drill stem tests, flow tests, production tests, or other tests meant to test fluids from specific intervals in a well or borehole), or other information matched to the measured magnetic susceptibility values that are collected from the one or more wells or boreholes that identify hydrogen source rock or intervals of potential hydrogen generation or accumulation as described in earlier embodiments may be extrapolated across a survey region for the purposes of identifying similar zones of hydrogen source rock or potential hydrogen generation or accumulation away from known datapoints (i.e., boreholes with magnetic susceptibility data).

As an example, extrapolation may involve mapping areas immediately around an individual well or borehole or multiple wells or boreholes from which this information is collected and is matched to magnetic susceptibility, and expanding the mapped intervals, magnetic bodies, or formations outward away from the wells or boreholes. In this manner, continuous source rock boundaries, boundaries of potential hydrogen generation, potential hydrogen reservoir boundaries, future exploratory or appraisal drilling targets, or potential “pay zones” may be mapped around the existing wells or boreholes within a survey area. Alternatively, extrapolation may not be continuous but instead may involve searching through other portions of the survey area for zones that have similar magnetic susceptibilities to the known lithologies or zones of interest as derived from the well or borehole data; this approach can be accomplished using cuttings or cores archived at various rock repositories stored from previously drilled wells.

As an example, in one embodiment extrapolation of high-resolution magnetic susceptibility data to calibrate magnetic and other geophysical models or improve magnetic field interpretations may involve mapping areas immediately around an individual well or borehole or multiple wells or boreholes from which this information is collected and is matched to magnetic susceptibility, and expanding the mapped intervals, magnetic bodies, or formations outward away from the wells or boreholes.

In an embodiment, measurements of magnetic susceptibility from one or more wells or boreholes (e.g., drill cuttings, core, or wireline data) may be integrated with other geophysical datasets (e.g., 2-D or 3-D seismic reflection, seismic refraction, seismic tomography, ambient noise tomography, ground-based, sea-based, or airborne gravity, gravity gradiometry, and electromagnetic) to refine and improve geophysical interpretations. This can be done by tying magnetic susceptibility data to magnetic field interpretations as described in earlier embodiments alone or in combination with other well or borehole data (e.g., drill cuttings description, XRD, XRF, SEM, hydrogen in mud gas, drill stem tests, flow tests, production tests, or other tests meant to test fluids from specific intervals in a well or borehole).

### Regional Methods to Measure Magnetic Susceptibility

Ground-based, sea-based, and airborne magnetometers, and more recently airborne magnetic gradiometers, are used to measure the magnetic field at discrete sampling points along parallel survey lines at regularly-spaced intervals (line spacing depending largely on desired spatial resolution) to create regional magnetic datasets. It is noted that ground-based and sea-based geophysical surveys may not involve parallel survey lines but irregularly distributed sampling points due to factors such as topography, and these should be considered only as examples. Magnetometers used for geological purposes generally include scalar and vector magnetometers. Scalar magnetometers measure the total magnetic field strength or intensity. Vector magnetometers utilize three sensors that detect the orthogonal (x, y, z) components of a magnetic field simultaneously, which determine the complete tensor of the magnetic field at a particular point. More recently, magnetic gradiometry has become favorable due to minimized regional effects, increased anomaly resolution, and the elimination of temporal variations.

In some embodiments, magnetic gradiometry involves multiple vector or scalar magnetometers separated by a fixed distance. Measurements by the magnetometers are made simultaneously and then compared to determine the rate of change in the magnetic field. The magnetic field data derived from these magnetometers can be used to indirectly calculate magnetic susceptibility of one or multiple magnetic bodies in the subsurface or as 3-dimensional magnetic susceptibility volumes through computer-based Werner deconvolution and geologic modelling.

Multiple techniques have been developed in the field of energy exploration as methods of calculating the depth to magnetic point sources based on regional magnetic field data, which are represented as magnetic anomalies, or a deviation from the average (or mean) magnetic field within a given geologic region or terrane. Two widely used techniques are Euler deconvolution or Werner deconvolution. Euler deconvolution is based on Euler's homogeneity equation and estimates source location and depth in cartesian (x, y, z) form using its ability to determine subtle changes in magnetization. The primary assumption is the structural index (SI), which may be summarized as how quickly the magnetic field decreases away from the source body, and the SI is influenced by the shape of the magnetic source body. Euler deconvolution does not require prior knowledge of magnetic susceptibility nor geological models to make interpretations. However, the Euler deconvolution method assumes that the subsurface geological structures have homogeneous petrophysical properties, which is an unreasonable assumption in many geological settings. The Euler deconvolution method also does not allow for the direct integration of real-world magnetic susceptibility data derived from the measurement of geological samples of interest.

To describe Werner deconvolution modelling, the Werner deconvolution is an example method of expressing a magnetic anomaly as a function of a magnetic source body's orientation, depth in the subsurface, and magnetic susceptibility at a specific point along a measured path orthogonal to the magnetic anomaly. Werner deconvolution uses an assumed geometry of the magnetic source body, such as a dike, prism, lens, cylinder, or basement horst, to calculate solutions that fit the measured magnetic intensity data. As an example, airborne magnetometers will measure magnetic field wavelengths and amplitudes. Computer-based Werner deconvolution may involve iteratively testing parameters that include: 1) magnetic source body geometry (both shape and orientation in the subsurface), 2) magnetic source body depth, and 3) magnetic susceptibility to find a best match to the measured magnetic wavelength and amplitude data (i.e., “inverse modelling”). Thus, to get regional magnetic interpretations, this process is repeated at discrete points along each parallel survey line and extrapolated for areas between the survey lines. The accuracy of the geologic models increases when the general regional geologic structure and type of magnetic bodies (e.g., dike, prism, lens, cylinder, or basement horst) are known or can be reasonably assumed.

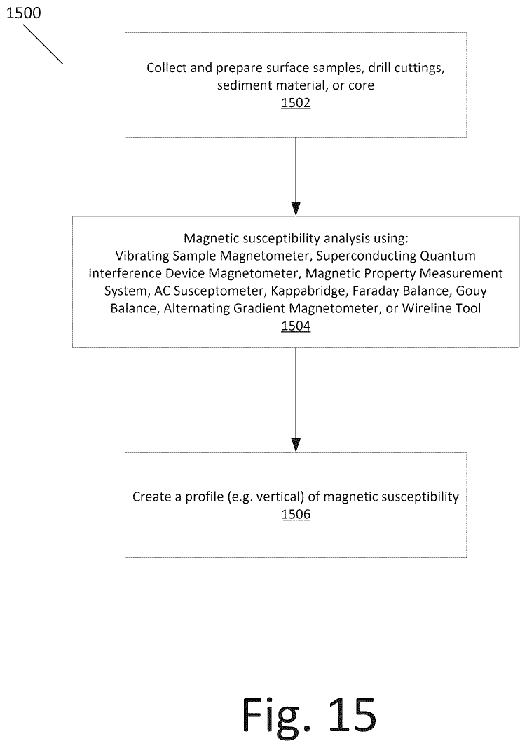

FIG. 15 illustrates an example method of creating profile data of magnetic susceptibility data. In a first step 1502, surface samples, drill cuttings, drilled whole core, sidewall cores, or sediment materials are collected and prepared as described in the following paragraphs. Direct magnetic susceptibility of the samples and at subsurface locations where the samples were collected are measured in step 1504. The instrumentation used to quantify magnetic susceptibility may include using a Vibrating Sample Magnetometer (VSM), Superconducting Quantum Interference Device (SQUID) magnetometer, Magnetic Property Measurement System (MPMS), AC susceptometer, Kappabridge, Faraday Balance, Gouy Balance, alternating gradient magnetometer, a cable-based downhole tool used to measure magnetic susceptibility in situ in a well or borehole, or other instrumentation or wireline tools. The profile is created in the third step 1506. The profile is used to identify intervals within the well or borehole favorable for hydrogen generation and accumulation and improve exploration for geologic hydrogen.

In one embodiment, the measurement of magnetic susceptibility in drill cuttings is used for exploration for geologic hydrogen. Samples of drill cuttings may be collected by an individual or automatically. For example, in drilling systems that utilize drilling mud, the cuttings may be collected at vibrating screens (shakers) below an ejection pipe that is used to separate solid materials from the rest of the drilling fluid. When air-drilling techniques are used, cuttings may be collected from the returning compressed air-or other gas-solids stream with or without the use of screens. Drill cuttings are frequently collected at regular intervals or when feasible during the drilling of a well or borehole, and geologic characteristics are described automatically or by an individual (mudlogger), which is in turn organized into a written log of rock descriptions and a sample catalog of rock materials across the depth of the drilled borehole. In this embodiment, regular or specific intervals of drill cuttings can be subsampled for measurement of magnetic susceptibility using one or more of the various measurement techniques described above.

In one embodiment, the measurement of magnetic susceptibility in cored rock or sediment is used for the purposes of improved exploration for geologic hydrogen. A core is a continuous section of geologic material obtained by drilling with a coring tool or bit. For example, core can be extracted during the drilling of a well or borehole using an advanced piston corer, rotary core barrel, diamond core barrel, or other. Cores can be extracted from a well or borehole wall as part of the drilling process or during post-drilling activities as sidewall core. Cores can also be drilled and extracted from a continuous rock body at the surface, or by drilling into previously sampled material (e.g., a core plug) through the use of smaller, often hand-held tools such as core drills. In this embodiment, regular or specific intervals of a core can be subsampled for measurement of magnetic susceptibility by various tools such as those described above.

In one embodiment, the measurement of magnetic susceptibilities along the wall of a well or borehole, performed by a tool lowered by cable (e.g., wireline tool), is used for the purposes of improved exploration for geologic hydrogen. The tool may be a standalone tool or as part of combination tools similar to, for example, triple-combo (e.g., gamma ray, bulk density, neutron porosity, resistivity) tools that are commonly used for industrial drilling for energy exploration or extraction. The tool may be used to generate a log of magnetic susceptibility values across depths (specific interval or entirety) of the well or borehole.

In one embodiment, the values of magnetic susceptibility or density can be used in combination with the mineralogical characteristics of samples collected from a well or borehole from drill cuttings. In this embodiment, the interpretations of lithologies that are described, for example, during mudlogging (e.g., cuttings description), derived by wireline logging (e.g., density, natural gamma radioactivity, sonic, or elemental spectroscopy), or measured by way of instrumentation such as XRD, XRF, SEM, ICP-MS, ICP-OES, or other device used to determine or infer mineralogy, lithology, or elemental composition from cuttings, core, or other discrete geologic samples.

In one embodiment, high-resolution magnetic susceptibility measurements may be used to identify prospective geological hydrogen source rocks, such as mafic and ultramafic rocks, and differentiate prospective geological hydrogen source rocks from other rock types with limited or no capacity to generate hydrogen. High-resolution magnetic susceptibility measurements may also be used to identify lithologic intervals that may serve as zones of hydrogen accumulation in a well or borehole. This may involve indirect identification of sedimentary units overlying mafic or ultramafic rocks, which constitute high-quality source rocks, or clastic weathering products of mafic rock that generally contain more magnetic minerals than granitic or sedimentary material. These lithologies have relatively elevated magnetic susceptibility that can be used to diagnose the distribution of natural hydrogen sources and volumetrically quantify the abundance of hydrogen generated from the formation of their composite minerals as shown in FIG. 1.

Notably, high magnetic susceptibility measurements are not signatures exclusive to mafic and ultramafic rocks. For example, banded iron formations (BIFs) are iron-rich sedimentary rocks that can exhibit magnetic susceptibility values between ˜0.01-5 SI units (see FIG. 1). Thus, an aspect of this embodiment includes the combination of magnetic susceptibility with other tools.

In instances where a well or borehole is logged with wireline tools, density and mineralogy characteristics may be used in combination with magnetic susceptibility to identify the presence of mafic and ultramafic rocks, to identify and differentiate rock types and mineral phases that result from the alteration of mafic and ultramafic rocks, or to differentiate mafic and ultramafic rocks from other rocks that are not capable of serving as natural hydrogen source rocks and to discretely evaluate the presence and relationship of these rocks along the depths of a well or borehole. One embodiment is the use of magnetic susceptibility in combination with wireline density, which is a wireline designed to continuously measure a formation's bulk density throughout the length of a borehole. Another embodiment is the use of magnetic susceptibility in combination with elemental spectroscopy (e.g., Litho Scanner® or LithoStar®) tools designed to estimate a formation's elemental composition throughout the length of the borehole.

### Integration of High-resolution Magnetic Susceptibility Data to Refine Regional Magnetic and Gravity Models

A common approach to interpreting ground-based, sea-based, or airborne magnetic field data is through Werner deconvolution inverse modelling using regularly sampled points along 2-dimensional survey lines. Regional surveys typically consist of many, evenly-spaced parallel survey lines, and Werner deconvolution results may be extrapolated through the spaces between adjacent lines to generate magnetic datasets across entire survey regions. Computer-based Werner deconvolution inverse modelling has been used to generate, for example, the contacts (i.e., depth to top), size, orientation, and magnetic susceptibility of one or more magnetic bodies across 2-dimensional or 3-dimensional space or to map the surface of crystalline basement. Additionally, similar approaches may be used to generate complete 3-dimensional volumes of magnetic susceptibility, which establish magnetic susceptibility values across all depths to the bottom of the modelled zone.

Despite innovations in automated or computer-based model interpretations, Werner deconvolution techniques require significant human interpretation, which is dependent on available knowledge of geologic structure and expected lithologies (i.e., magnetic susceptibilities) in the region of interest. In particular, the non-unique solutions of the Werner deconvolutions need to be evaluated within the context of the expected structure, sedimentary stratigraphy and thickness, and character of any magnetic bodies to determine if the solutions are reasonable or constitute plausible geologic scenarios. Werner deconvolution capabilities are also dependent on data resolution (e.g., line spacing, type of magnetometer).

As an example, if a relatively thin magnetic dike overlies a massive magnetic body, the massive magnetic body may overwhelm the measured magnetic field signal, making it difficult or impossible to detect the smaller magnetic body. Further, magnetic bodies present below a relatively massive magnetic body are similarly difficult to distinguish. When no prior data are available, many interpretations also use general assumptions about expected magnetic susceptibilities of lithologies such as the low magnetic susceptibility of sedimentary rocks compared to most crystalline rocks, despite the possibilities of iron-rich sediments (see FIG. 1). As a result of these limitations, one embodiment of the current disclosure is improved magnetic field data interpretation methods for regional ground-based, sea-based, and airborne aeromagnetic surveys. This embodiment also improves the ability to integrate other data types (e.g., petrophysical, seismic) in order to develop reconcilable geologic models that incorporate available data prior to geologic hydrogen exploration.

By way of example, two methods of tying well or borehole magnetic susceptibility data to regional magnetic data will be described. It is noted that the methods described herein do not preclude other methods of associating well or borehole magnetic susceptibility data to regional magnetic survey data, collected by either ground-based, sea-based, or airborne magnetic methods (see FIG. 15). Two methods of creating a magnetic well tie include: 1) using Werner deconvolution inverse modelling as described above, in manners that may include selecting various solutions of depths and magnetic susceptibilities that may be generated during the iteration process that best match the measured depths and magnetic susceptibilities across the well or borehole as the correct solution at that location, or by using other inverse modelling techniques or 2) by using the measured depth and associated magnetic susceptibilities of the lithologies across the well or borehole as known values and using the Werner deconvolution to generate a model that fits the measured data (known as “forward modelling”), or by using other similar forward modelling techniques.

In the first approach, inverse models use the measured magnetic field data from the magnetometer as a known and generate possible solutions of magnetic source depth and magnetic susceptibility. When using this approach, the solution is selected based on the best match to the well or borehole data (i.e., based on total sum of squares (TSS) from least-squares QR (LSQR) where Q is used to describe the orthogonal matrix and R is used to describe the triangular matrix or other solution algorithms). In the forward model approach, the model is generated based on the airborne magnetic field measurements and at least one other known (geometry or magnetic susceptibility of the magnetic body). When solving the forward problem in magnetics, the geometry of the magnetic anomaly generally has the greatest amount of uncertainty (e.g., magnetic susceptibility is more easily assumed) without additional subsurface data (e.g., seismic), and the shape, extent, and volume of the feature can be adjusted to improve agreement with the measured magnetic field data.

Alternatively, as an example, forward modelling may instead involve generating a model that uses the measured well or borehole magnetic susceptibility data from one or more wells or boreholes to solve for a magnetic source body geometry and picking the solution that provides the best match to the magnetic field data measured by the magnetometer (i.e., setting depth and magnetic susceptibility as fixed values and comparing the values of the calculated magnetic field to the airborne measurements). The desired outcome of either modelling approach is a geologic map or model of magnetic susceptibilities in the subsurface across a 2-dimensional cross-sectional area or 3-dimensional volume that matches the magnetic susceptibilities and lithological segmentations or stratifications at the borehole location along the cross-section planes and across the depth of the borehole, similar to a seismic well tie within a 2-D seismic section or intersecting 3-D seismic slices.

In another embodiment, the magnetic interpretations that utilize magnetic susceptibilities from one or more wells or boreholes, may be integrated with other potential field data. Airborne, sea-based, or ground-based magnetic or magnetic gradiometry surveys are commonly combined with airborne gravity or gravity gradiometry (AGG), airborne gravity and magnetics (AGM), ground-based gravity and magnetics, or sea-based gravity and magnetics, or ground-based or airborne electromagnetic (EM) surveys. While not essential, gravity or gravity gradiometry data in particular provide useful information to help identify hydrogen source rocks (e.g., mafic and ultramafic rocks) due to increased density values commonly associated with these rocks compared to background geologic materials (see FIG. 2). The magnetic susceptibility of hydrogen source rocks may overlap with other materials (see FIG. 1), and gravity data can provide an external tool to aid in the identification of potential hydrogen source rocks, zones of hydrogen generation, or zones of hydrogen accumulation.

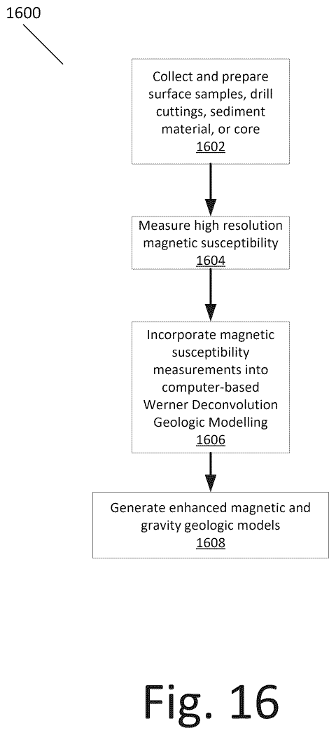

FIG. 16 illustrates a method of associating well or borehole magnetic susceptibility data to regional magnetic survey data, collected by either ground-based, sea-based, or airborne magnetic methods using Werner deconvolution modelling. In a first step 1602, surface samples, drill cuttings, drilled whole core, sidewall cores or sediment materials are collected and prepared as described herein. High-resolution magnetic susceptibility of the samples is measured in step 1604. The magnetic susceptibility measurements are incorporated into the Werner deconvolution modelling in the third step 1606. In a fourth step 1608, enhanced magnetic and gravity geologic models are generated.

By way of example, integrating gravity data with the refined magnetic methods described in earlier embodiments may include direct comparisons and adjustment of interpreted lithologic or mineralogic boundaries in 2-dimensional or 3-dimensional space. Gravity or gravity gradiometry data may be interpreted using computer-based Werner deconvolution inverse or forward modelling in similar fashions to magnetic data, producing analogous density models in 2-dimensional cross sections or 3-dimensional volumes. Variable alteration can lead to differing magnetic susceptibilities within the same formation, and the alteration of hydrogen source rocks may change the density of the materials (e.g., serpentinization leads to a volume increase with lower resulting density while the oxidation of iron leads to increased magnetic susceptibility). Therefore, having a refined magnetic model that can be compared to density models may provide more confidence in the geologic model interpretations as well as reveal other valuable information.

Similar methods may be used to compare magnetic models, or integrated gravity and magnetic models, with electromagnetic data. Electromagnetic (EM) surveys are conducted by inducing a magnetic field and measuring the responses, which are influenced by subsurface materials. EM is useful in detecting conductive materials in the subsurface, which can be influenced by lithology, pore water salinity, or presence of conductive minerals (e.g., copper sulfides). EM surveys have been used in prior art for identifying serpentinized zones within ultramafic rock. Therefore, there is a utility in integrating EM data with magnetics or AGM as an additional tool to refine the interpretation of hydrogen source rocks or zones of potential hydrogen generation within hydrogen source rocks. However, generally the depth of investigation for EM is relatively shallow (e.g., 500-600 m), and would have greater application in near surface hydrogen generation (or carbon or sulfur mineralization) targets.

In this embodiment, the refined geologic models developed from the computer-based Werner deconvolution inverse or forward modelling of magnetic field data, which utilize magnetic susceptibility measurements from one or more wells or boreholes as described in earlier embodiments, alone or following integration with other potential field data (e.g., AGM, EM), may be used to refine the interpretation of 2-D or 3-D seismic reflection or refraction seismic data, or seismic tomography data to generate a more parsimonious geologic interpretation across a survey area. Seismic reflection utilizes the generation of sound waves and measuring changes in acoustic impedance to image subsurface geological information. Acoustic impedance is largely influenced by density contrasts, but may change based on, for example, changes in mineralogy, lithology (e.g., shale compared to carbonate, sedimentary rock compared to igneous rock), porosity, or fluid composition. As a result, seismic reflection data is capable of imaging stratigraphic changes, igneous intrusions, crystalline basement, faults, and fracture zones as well as impedance changes due to gas or oil accumulations.

Seismic reflection has been used to differentiate crystalline bodies from surrounding sedimentary rock due to the generally large difference in seismic velocities between sedimentary (P-wave velocities generally <4.5 km/s with the exception of evaporites or carbonates which may be up to ˜6.5 km/s) and crystalline rocks (P-wave velocities generally >4.5 km/s and up to >8.0 km/s depending on mineralogy and pressure conditions). Prior art has not involved differentiating hydrogen exploration related materials (e.g., mafic and ultramafic rocks) from other material (e.g., granitic or non-mafic metamorphic basement), or imaging zones within crystalline rocks that may have undergone alteration or hydrogen generation.

Mafic or ultramafic igneous rocks, or mafic or ultramafic metamorphosed rocks, are generally more dense than granitic igneous rocks or granitic metamorphosed rocks (FIG. 2). Therefore, sound waves generally travel faster through mafic and ultramafic rocks compared to intermediate or granitic rocks that are comparatively silica-rich and much lower in magnesium and iron. However, the difference in seismic velocity between these materials may be relatively small or non-existent depending on the mineralogies of the specific rocks in question and extent and type of alteration. For example, while the seismic P-wave velocities of ultramafic rocks may be >7 km/s, mafic rocks such as basalt or gabbro may have velocities ranging from ˜5.5-6.5 km/s. In comparison, the seismic P-wave velocities of granitic rocks may range from ˜4.5-6 km/s. Increasing grades of metamorphism (e.g., FIGS. 9 and 10) may also lead to increasing seismic P-wave velocities.

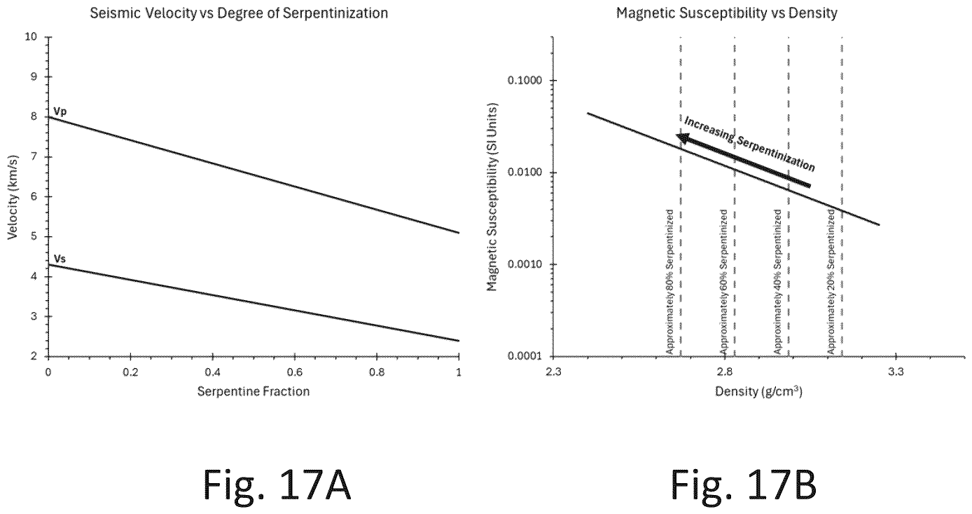

In contrast, the seismic P-wave velocities of serpentine may decrease slightly from the original unaltered rock due to volume expansion (V_{P}˜5.15±0.45 km/s depending on starting composition and degree of alteration), but can be distinguished more clearly based on the significant decreases in density (serpentine ρ˜2.6 g/cm³, basalt ρ˜3.0 g/cm³) and shear (S-wave) velocity (serpentine V_{S}˜2.6+0.2 km/s, basalt V_{S}˜3.2±0.4 km/s). The combination of these effects can produce a unique V_{P}/V_{S} that often exceeds 2. Because serpentinization will produce magnetite during the reaction of olivine and pyroxene, the elevated magnetic susceptibility values of these altered lithologies can serve as an additional constraint to identify zones of altered lithology relevant to hydrogen generation and improve hydrogen exploration efforts by uniquely identifying zones of past serpentinization, which is not necessarily feasible using seismic data alone (FIG. 17).

Further, the effects of fractional crystallization in igneous rocks of interest on acoustic impedance would largely be unconstrained without additional data. Therefore, without specific data (e.g., from the well or borehole or through magnetic surveys), seismic reflection surveys often cannot be used to resolve compositional differences between hydrogen source rocks and other crystalline rocks.

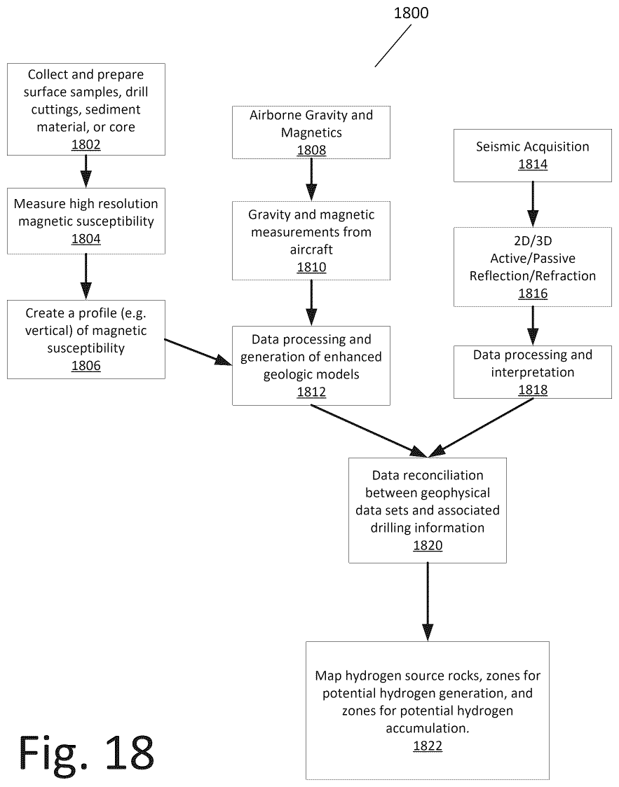

FIG. 18 illustrates an example method of associating well or borehole magnetic susceptibility data to regional magnetic survey data, collected by either ground-based, sea-based, or airborne magnetic methods using Werner deconvolution modelling. The method illustrated in FIG. 18 builds on and refines the method set forth in FIG. 16. Steps 1802, 1804, and 1806 correspond to steps 1602, 1604, and 1606 described above. Further, AGM data and gravity and magnetic measurements from an aircraft are received in steps 1808 and 1810 and incorporated into the geologic model in step 1812. Additionally, seismic data and 2D/3D reflection or refraction data is received in steps 1814 and 1816. These data are processed and interpreted in step 1818.

In step 1820, the geophysical results from steps 1812 and 1818 are reconciled with the magnetic susceptibility data of step 1806. The hydrogen source rocks, zones of potential hydrogen generation and zones of potential hydrogen accumulation are mapped in step 1822.

### Magnetic Susceptibility to Improve Airborne and Gravity Magnetics (AGM) for Hydrogen Exploration

In this embodiment, the refined geologic models developed from the computer-based Werner deconvolution inverse or forward modelling of magnetic field data, which utilize magnetic susceptibility measurements from one or more wells or boreholes as described in earlier embodiments, alone or following integration with other potential field data such as AGM or EM, when integrated with 2-D or 3-D seismic reflection data may be used to: 1) identify lithological zones within the seismic sections that correspond to hydrogen source rocks, or zones within those source rock bodies that may correspond to potential hydrogen generation or hydrogen accumulation, or additionally identify overlying formations that may potentially serve as reservoirs and seals for a hydrogen accumulation if the seismic data is of sufficient quality; or 2) identify lithological zones within the seismic sections that correspond to hydrogen source rocks. It is noted that the geologic models developed from the magnetic data, or possibly magnetic and gravity or magnetic, gravity, and EM data, are exported in depth units while raw seismic data are in two-way travel time, therefore, a seismic well tie should be conducted to convert the seismic data from two-way travel time to depth before integrating the aforementioned geologic models.

Additionally, the refined 3-dimensional geologic models developed from the magnetic susceptibility data, or possibly magnetic and gravity or magnetic, gravity, and EM data that utilize the magnetic susceptibility measurements from one or more wells or boreholes may be used to supplement 2-D seismic reflection data within the survey area. AGM surveys in prior art have been used to supplement or replace seismic data as a low-cost alternative in certain scenarios due to the relatively efficient ability for AGM data to map, for example, crystalline basement structure and some major density contrasts (e.g., carbonate formations). Crystalline basement is often mapped assuming that the crystalline basement will generally display a significant magnetic susceptibility increase compared to overlying sedimentary rocks or sediment (see FIG. 1), while methods to identify density contrasts utilize the gravity or gravity gradiometry component of the AGM surveys often exclusively. The Werner deconvolution models were interpreted by simply overlaying the seismic and magnetic field data. Prior art may use interpreted seismic data or borehole data (e.g., mud log cuttings descriptions) within the survey areas for comparisons to the geologic models that have been generated. These may, for example, compare the modelled depth to crystalline basement using AGM with the seismic interpretation or the presence and morphology of intrusions. Prior art may use knowledge of geologic information from drilled wells or boreholes (e.g., drill cuttings or core descriptions) within a survey area to evaluate the quality of a geologic model. In this disclosure, a 3-dimensional geologic model is used to supplement 2-dimensional seismic data more adequately through the ability to perform both a seismic well tie and magnetic susceptibility tie to the same one or more boreholes. This approach provides the ability to map hydrogen source rocks and overlying formations that may potentially serve as hydrogen reservoirs or sealing lithologies because this approach allows certain acoustic impedance horizons on the seismic data interpretations to be matched to specific magnetic susceptibilities.

The spatial resolution of the geologic models developed from the magnetic data, or possibly magnetic and gravity or magnetic, gravity, and EM data as described in earlier embodiments are generally lower compared to seismic data. Higher magnetic susceptibility zones in the models may appear as “blobs” whereas in reality they may be comprised of, for example, multiple discrete layers or flows (i.e., in the case of mafic igneous rock) that may be visible in the seismic data as parallel and alternating acoustic impedance contrasts. Therefore, in this embodiment a final geologic model may be developed that integrates all available data for a best fit, including the magnetic susceptibility measurements from the one or more wells or boreholes that are included in the magnetic susceptibility geologic model, seismic data, other petrophysical data, gravity data, EM data, mineralogical data, or hydrogen in mud gas, drill stem tests, flow tests, production tests, or other tests meant to test fluids from specific intervals in a well or borehole.

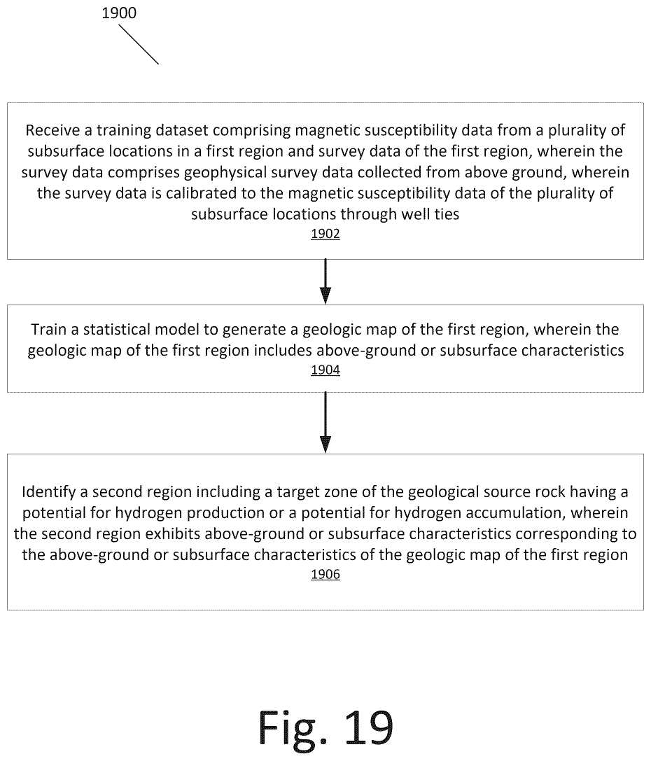

As an example, FIG. 19 illustrates a method for utilizing magnetic susceptibility data combined with geophysical survey data in one region to identify geological hydrogen source rock in other regions. In a first step 1902, a training dataset is received for training a statistical model or algorithm. The training dataset includes magnetic susceptibility data from a plurality of subsurface locations in a first region as well as survey data of the first region. The subsurface locations include locations within igneous rock, metamorphic rock, or mafic mineral-or iron-rich sedimentary rock in the region. The survey data includes geophysical survey data collected from above ground calibrated to the magnetic susceptibility data of the plurality of subsurface locations through well ties.

In a second step 1904, the statistical model is trained using the training dataset to generate a geologic map or model of the first region. The geologic map or model of the first region includes characteristics that may be used in a subsequent step to identify regions including rock formations that may be used to generate hydrogen and/or store hydrogen accumulation. Examples of such characteristics include magnetic susceptibility data, gravity data, density data, mineralogical data, and combinations thereof.

In a third step 1906, a second region is identified using the statistical model. The second region includes a target zone of the geological hydrogen source rock having a potential for hydrogen production or a potential for hydrogen accumulation. The second region is identified based on characteristics that it exhibits and that correspond to the characteristics of the geologic map or model of the first region.

### Magnetic Susceptibility to Improve Economic Estimates and Reduce Exploration Costs

A geologically plausible 2-D or 3-D geologic model over a survey area that integrates measurements of magnetic susceptibility from one or more wells or boreholes may provide known or calculated spatial distribution and volumetric estimates of discovered hydrogen reservoirs or potential additional reservoirs. Volumetric calculations for a prospect or area of interest allows for gas in place (GIP) and estimated ultimate recovery (EUR) predictions when combined with gas compositional data.

In an embodiment, the measurements of magnetic susceptibility from a well or borehole (e.g., drill cuttings, core, wireline data) may be used to define the scope of geophysical (e.g., AGM, 2-D or 3-D seismic) surveys. If magnetic susceptibility data can be obtained for multiple boreholes, a specific interval or feature relevant to hydrogen exploration may be identified in some or all boreholes, which can restrict the geographic or vertical extent of the planned geophysical data acquisition, focusing the results and reducing unproductive acquisition and processing costs.

In an embodiment, the magnetic, integrated magnetic and gravity, or integrated magnetic and electromagnetic geologic models that include the magnetic interpretations derived from the magnetic susceptibility measurements from one or more wells or boreholes (e.g., drill cuttings, core, wireline data), and generated through the computer-based Werner deconvolution modelling as described in earlier embodiments, may be used to constrain seismic reflection survey areas. The extrapolated magnetic susceptibility data within the refined geologic models may identify hydrogen source rocks, or zones of potential hydrogen generation or accumulation across a region of interest. The results of these geologic models may define target formations or zones in 2-dimensional or 3-dimensional space, which may then warrant seismic reflection surveys to acquire high resolution 2-dimensional or 3-dimensional spatial constraints of these target zones or bodies, as well as identify faults or other potential hazards, or generate a geoprognosis in preparation for the drilling and exploitation of these resources.

In an embodiment, measurements of magnetic susceptibility from material at or near the surface, used alone or in combination with other tools (e.g., XRD, XRF, SEM), may be used to define a surface magnetic susceptibility value that may then be included in the computer-based inverse or forward modelling as described in earlier embodiments, may be removed during the interpretation of magnetic field data as background, or used to identify potential hydrogen source rocks or the surface expression of zones of alteration, faults, or fracturing, or potentially hydrogen generation within hydrogen source rocks. It is noted that surface weathering processes frequently affect and alter rocks, sediments, and other geologic material at the surface, and this weathering zone may extend centimeters, meters, or tens of meters into the subsurface.

This embodiment may involve digging, boring, the use of soil probes, shallow core drilling, or other tools used at the surface to reach rock, sediment, or other geologic materials that have not been affected by surface weathering processes. Surface magnetic susceptibility measurements may be particularly important if there are lithologies at or near the surface with distinct magnetic susceptibilities, such as in areas where banded iron formations are at or near the surface or where massive volumes of ultramafic rock exist at or near the surface (e.g., ophiolite complexes). Additionally, in this embodiment, measurements of magnetic susceptibility from magnetic bodies (e.g., hydrogen source rocks) exposed at or near the surface may also be used to identify surface expressions of zones of increased alteration (e.g., from serpentinization, oxidation, metamorphism, or other alteration processes). By way of example, a regularly-spaced sampling transect across the surface of an ophiolite exposed at the surface may be able to identify relative changes in magnetic susceptibility from a baseline value. Such changes in magnetic susceptibility from a baseline value may be indicative of alteration processes such as increased oxidation to hematite, suggesting a lesser-or non-hydrogen generating reaction, or increased serpentine and magnetite, indicating a potential zone of hydrogen generation. Discrete zones of alteration and changes in magnetic susceptibility at or near the surface may also represent the surface expressions of faults or fracturing, which may act as migration pathways both for fluid introduction (i.e., oxygen-rich groundwater) as well as natural gas escape to the atmosphere. If identified, these features can then be mapped through further surface mapping or by using subsurface data (e.g., reflection seismic, refraction seismic, or seismic tomography) to better evaluate exploration or drilling risks or targets.

In an embodiment, the magnetic, integrated magnetic and gravity, or integrated magnetic and electromagnetic geologic models that include the magnetic interpretations derived from the magnetic susceptibility measurements from one or more wells or boreholes (e.g., drill cuttings, core, wireline data), and generated through the computer-based Werner deconvolution modelling as described in earlier embodiments, alone or when integrated with other boreholes (e.g., wireline logs, XRD, XRF, SEM) or subsurface data (e.g., 2-D or 3-D seismic) may be used to identify, evaluate, score, and rank conventional or unconventional geologic hydrogen systems.

When exploring for “conventional” natural hydrogen targets, hydrogen source rocks are identified beneath sedimentary formations that have sufficient porosity and permeability to serve as hydrogen reservoirs (e.g., sandstones, porous carbonates), which are in turn capped by relatively impermeable seals (e.g., salt, anhydrites, other evaporites, low permeability shales, or unfractured igneous rocks), and these formations are in a stratigraphic or structural configuration so as to create a 3-dimensional trapping structure that may serve as a zone of accumulation. The possibilities of hydrodynamic trapping where hydrogen gas is trapped by flowing groundwater (i.e., not necessarily physically above the hydrogen source rock) are also considered. Ideal geologic hydrogen source rocks will have magnetic susceptibility values that are well in excess of surrounding crustal rock (e.g., granite, sandstones, carbonate) and elevated compared to unaltered hydrogen source rock (e.g., basalt, gabbro, dunite). Mature source rocks that generate hydrogen in conventional systems will contain high amounts of altered mineral phases (e.g., magnetite, hematite) and be represented by a combination of relatively high density and magnetic susceptibility values in regional AGM models. Seismic velocities for these hydrogen source rocks will also be elevated (e.g., V_{P}˜5-6 km/s) compared to surrounding rock, but may exhibit a higher V_{P}/V_{S} value resulting from the significant drop in V_{S} associated with serpentinization (FIG. 17).

In this embodiment, the magnetic susceptibility measurements, alone or integrated into the refined geologic models as described above, may be used to identify potential conventional reservoir and/or sealing formations for natural hydrogen accumulations in addition to identifying hydrogen source rocks. The high-resolution magnetic susceptibility measurements may distinguish certain sedimentary or igneous formations that have sufficiently different magnetic susceptibility from the surrounding formations, some of which may act as reservoirs or seals. In such scenarios, these magnetic susceptibility contrasts may be mappable, for example, from one borehole to adjacent boreholes, or regionally through the integration of geophysical survey data and Werner deconvolution modelling as described above. As an example, basal sandstones, particularly those overlying mafic crystalline basement, often contain elevated levels of iron-oxide minerals which increase magnetic susceptibility. Further, these types of formations often have sufficient porosity and permeability to be potential reservoir rocks for conventional hydrogen accumulations. As another example, in some settings igneous sills that have intruded into sedimentary rocks may act as seals for natural hydrogen accumulations (e.g., of migrated hydrogen that was generated from a deeper source rock), and the igneous rock may be sufficiently magnetic to be distinct from surrounding geologic material. Integrating the magnetic susceptibility measurements and/or the refined geologic models with other data (e.g., gravity, seismic, mineralogy, petrophysics) will further improve the ability to map formations that may potentially serve as hydrogen reservoirs or seals. In geologic settings where the hydrogen source rock and potential reservoir-seal combinations can be mapped, either based on magnetic susceptibility alone or through integration of other data types, the degree of alteration of the hydrogen source rock (i.e., potentially indicating extent of hydrogen generation) based on magnetic susceptibility may be used to make economic assessments of individual hydrogen systems.

One embodiment develops a method for identifying, evaluating, scoring and ranking high-quality source rocks using the methods disclosed above for “conventional” hydrogen exploration based on measuring and identifying rocks with a high initial proportion of primary mafic minerals on a volumetric basis that have a present mineralogy dominated by secondary mineral phases. Additional and standard exploration workflows are required to identify specific targets for conventional natural hydrogen exploration in these geologic settings based on mapping migration pathways, prospective reservoirs, prospective trapping geometries, and prospective sealing lithologies. Importantly, this embodiment can be used to score and rank prospective hydrogen systems at various scales. For illustrative purposes, this can include on global scales, within a particular region, within a country, within a basin or system, or even within a borehole.

In “unconventional” natural hydrogen exploration, hydrogen source rocks are identified and zones within those source rock bodies are targeted for in situ accumulations of hydrogen. The reservoirs in “unconventional” hydrogen systems consist of intervals of natural open fractures (i.e., fracture porosity) that are sealed by less fractured intervals, intervals with micro or nanoporosity (e.g., generated due to alteration processes), or as intervals that may be exploited using hydraulic fracturing to release previously trapped hydrogen. In this manner, the hydrogen source rocks may serve as sources, reservoirs, and seals roughly analogous to organic-rich shales or carbonates that have been exploited as “unconventional” oil and gas plays through the use of horizontal drilling and hydraulic fracturing. High-quality source rocks may be identified through the methods defined in earlier embodiments. Within identified hydrogen source rocks, zones of interest for unconventional exploitation may show localized, relative changes in magnetic susceptibility that may represent more altered zones (e.g., hydrated, serpentinized, metamorphosed) where hydrogen may have been generated. Associated changes in rock densities may accompany these processes depending on the products generated. For example, the serpentinization process leads to an increase in magnetic susceptibility due to the generation of magnetite and decrease in density due to an increase in rock volume. Changes in volume within the rock body may also lead to increased natural fracture generation caused by the process of serpentinization. Alternatively, the alteration of silicate minerals to clay may also develop porosity in what was originally low porosity igneous or metamorphic rocks. Changes in rock density due to hydrogen generation, other mineral alteration, or fracturing may be identifiable using AGM when used in combination with other subsurface data (e.g., 3-D seismic). Thus, the identification of “unconventional” targets may involve using AGM techniques as described in earlier embodiments, alone or in combination with other subsurface data (e.g., seismic, tomography, ambient noise tomography, mineralogy), to identify zones within hydrogen source rocks of potential alteration, increased porosity, or increased hydrogen generation that would serve as targets for “unconventional” hydrogen exploration. One embodiment develops a method for identifying, evaluating, scoring and ranking high-quality source rocks using the methods disclosed above for “unconventional” hydrogen exploration based on measuring and identifying rocks with a high initial proportion of primary mafic minerals on a volumetric basis that have a present mineralogy dominated by secondary mineral phases. Additional and standard exploration workflows are required to identify specific targets for “unconventional” natural hydrogen exploration in these geologic settings based on mapping migration pathways, prospective reservoirs, prospective trapping geometries, and prospective sealing lithologies. Importantly, this embodiment can be used to score and rank prospective hydrogen systems at various scales. For illustrative purposes, this can include on global scales, within a particular region, within a country, within a basin or system, within a borehole, within a given lithology, or sublithology.

In some geologic settings, hydrogen generated in one location of a hydrogen source rock may migrate through intra-source rock fractures, faults, or previously generated microporosity due to earlier alteration events, and accumulate in a second location of the same source rock body that was previously not charged with hydrogen. In so doing, the hydrogen accumulates in relatively porous and permeable zones that are contained by relatively low porosity and permeability zones in analogous manners to “conventional” hydrogen exploration. However, the source rock-reservoir may be exploited using techniques used in “unconventional” hydrogen exploration (e.g., horizontal drilling and hydraulic fracturing). This may occur, for example, in relatively massive or thick mafic or ultramafic deposits (e.g., large igneous provinces such as the Columbia River Basalt, Deccan Traps, or Siberian Traps; ophiolite complexes) that are susceptible to localized alteration of varying degrees. As examples, variable alteration may occur based on migration pathways for groundwater through hydrogen source rocks, (e.g., where faults or localized fracture zones facilitate relatively increased water-rock interactions in localized areas). This may be of particular importance in areas of hydrothermal alteration where heated water is responsible for the alteration as opposed to, for example, regional metamorphism where heat may be relatively uniform across large areas. Alternatively, relatively broad water infiltration may still lead to variable amounts of alteration depending on the ambient temperatures (e.g., geothermal gradient). For example, serpentine (and therefore hydrogen) may be generated at greater depths within a source rock formation where the ambient temperature and pressure conditions are ideal for serpentinization, while alteration at lower temperatures (e.g., at shallower depths) may lead to the preferential formation of brucite, which may still increase porosity due to mineral breakdowns as described above but produce little or no hydrogen. In other scenarios, stacked lava flows may experience variable alteration due to differing amounts of time specific lava flows are exposed at the surface. Further, alteration due to weathering processes at or near the surface generally will not produce hydrogen due to the abundance of free oxygen.

If hydrogen is generated in systems with variable alteration (e.g., after burial by subsequent lava flows), the alteration due to hydrogen generation may open new migration pathways as microporosity or new fractures as described above. As a result, hydrogen may migrate from the location of generation (e.g., buoyantly up dip) to other porous intervals that may have increased porosity but as a result of non-hydrogen forming processes. In these scenarios, the magnetic susceptibility may be higher in the zones of hydrogen generation as described in previous embodiments, but the zone of accumulation may have only moderate, little, or no relative change in magnetic susceptibility. In such scenarios, integrating other data types (e.g., formation or cuttings gas) may aid in the identification of hydrogen accumulations. Additional exploration workflows are required to identify specific targets for hydrogen that has migrated but accumulates in source rock-reservoirs that would necessitate “unconventional” natural hydrogen exploration techniques. These may include mapping migration pathways, prospective reservoirs, prospective trapping geometries, and prospective sealing lithologies, and analyzing formation fluids. Importantly, this embodiment can be used to score and rank prospective hydrogen systems at various scales. For illustrative purposes, this can include on global scales, within a particular region, within a country, within a basin or system, within a borehole, within a given lithology, or sublithology.

### Low Carbon Intensity Hydrogen

There is a significant focus today on the decarbonization of energy and chemical industries to positively impact climate change. In response, companies and individuals are actively working to produce cost-effective “clean” or “green” hydrogen and other chemicals. Hydrogen is labelled as “green” when its production results in significantly lower greenhouse gas emissions compared to the production of other energy sources. Governments have recently begun to categorize hydrogen by assessing the emissions intensity of the production plant or system from which the hydrogen is sourced. Specifically, components or portions of the hydrogen gas production process, including the hydrogen feedstock from a wellhead as well as the hydrogen gas product, can be assigned a carbon intensity (CI) score according to the greenhouse gas emissions resulting from the particular component or portion. The CI scores referenced herein are provided in kg CO₂ equivalent greenhouse gases per kg H₂ produced (kg CO₂eq/kg H₂).

In some embodiments, the target zones of the geologic hydrogen source rock identified by the methods and systems described herein as suitable sources for hydrogen exploration and/or hydrogen accumulation provide a feedstock including hydrogen gas. Feedstock extracted from boreholes into the subsurface from wellheads may be of a sufficient composition that it can be subsequently separated and/or purified to between about 90% and about 99.9999% purity, meeting the needs of the hydrogen markets.

In some examples, the feedstock includes primarily hydrogen gas. The feedstock may also include additional gas constituents such as nitrogen, carbon dioxide, methane and noble gases such as helium, neon, argon, krypton, xenon, or radon. In some examples, the feedstock has a CI score of less than 4.0 kg CO₂eq/kg H₂, or less than 3.0 kg CO₂eq/kg H₂, or less than 1.5 kg CO₂eq/kg H₂, or less than 0.45 kg CO₂eq/kg H₂. For example, a feedstock that includes at least 50 mol %, 60 mol %, 70 mol %, 75 mol %, 80 mol %, 85 mol %, 90 mol %, 95 mol %, 98 mol %, or 99% mol % hydrogen, less than 15 mol %, 12.5 mol %, 10 mol %, 9 mol %, 8 mol %, 7 mol %, 6 mol %, 5 mol %, 4 mol %, 3 mol %, 2 mol %, 1 mol %, 0.5 mol %, or 0.1% mol % carbon dioxide, less than 12.5 mol %, 10 mol %, 9 mol %, 8 mol %, 7 mol %, 6 mol %, 5 mol %, 4 mol %, 3 mol %, 2 mol %, 1 mol %, 0.5 mol %, 0.1 mol % methane (CH₄), and up to 50 mol %, 45 mol %, 40 mol %, 35 mol %, 30 mol %, 25 mol %, 20 mol %, 15 mol %, 12.5 mol %, 10 mol %, 9 mol %, 8 mol %, 7 mol %, 6 mol %, 5 mol %, 4 mol %, 3 mol %, 2 mol %, or 1 mol % nitrogen provides a CI score of less than 4.0, 3.0, 1.5, or 0.45 kg CO₂eq/kg H₂. In such cases, the hydrogen produced or obtained from these geological sources may be classified as low carbon intensity hydrogen.

### Conclusion

It is noted that there is no requirement to provide or address the theory underlying the novel and groundbreaking systems, methods, performance or other beneficial features and properties that are the subject of, or associated with, embodiments of the present disclosure. Nevertheless, various theories are provided in this specification to further advance the art in this critical area, and in particular in the important area of hydrogen, dihydrogen sulfide, carbon dioxide, and helium exploration, production, and downstream conversion or utilization. These theories put forth in this specification, and unless expressly stated otherwise, in no way limit, restrict or narrow the scope of protection to be afforded the claimed embodiments. It is further understood that the present disclosure may lead to new, and heretofore unknown theories to explain the conductivities, drainages, resource production, chemistries, and function-features of embodiments of the methods, articles, materials, devices, and systems of the present disclosure and that such later developed theories shall not limit the scope of protection afforded the present disclosure. Other embodiments than those specifically disclosed herein may be included without departing from its spirit or essential characteristics. The embodiments disclosed are to be considered in all respects only as illustrative and not restrictive. The various aspects and embodiments disclosed herein are for purposes of illustration and are not intended to be limiting. The various embodiments of devices, systems, activities, methods, and operations set forth in this specification may be used with, in, or by, various processes, industries, and operations, in addition to those embodiments of the Figures and disclosed in this specification. The various embodiments of devices, systems, methods, activities, and operations set forth in this specification may be used with other processes, industries, and operations that may be developed in the future; with existing processes, industries, and operations, which may be modified, in-part, based on the teachings of this specification; and with other types of gas recovery and valorization systems and methods.

Further, the various embodiments of devices, systems, activities, methods, and operations set forth in this specification may be used with each other in different and various combinations. Thus, the configurations provided in the various embodiments of this specification may be used with each other. For example, the components of an embodiment having A, A′, and B and the components of an embodiment having A″, C, and D can be used with each other in various combination (e.g., A, C, D, and A; A″, C, and D; etc.) in accordance with the teaching of this specification. Thus, the scope of protection afforded by the present inventions should not be limited to a particular embodiment, configuration or arrangement that is set forth in a particular embodiment, example, or in an embodiment in a particular Figure. Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

1. A method for accessing a target zone of geological hydrogen source rock at a region for improved exploration of geologic hydrogen, the method comprising:
collecting magnetic susceptibility data from a plurality of subsurface locations within the geological hydrogen source rock in the region, wherein the plurality of subsurface locations includes subsurface locations in igneous rock, metamorphic rock, or mafic mineral-or iron-rich sedimentary rock within the region,
identifying a magnetic anomaly in the magnetic susceptibility data wherein the magnetic anomaly is at a subsurface location above, below or within the igneous rock, the metamorphic rock, or the mafic mineral-or the iron-rich sedimentary rock, wherein the igneous rock, the metamorphic rock, or the mafic mineral-or the iron-rich sedimentary rock includes the target zone of the geological hydrogen source rock having a potential of hydrogen production without injection of a reactant or a potential of hydrogen accumulation;
receiving geophysical survey data of the region, wherein the geophysical survey data comprises geophysical data collected above ground;
tying the magnetic susceptibility data to the geophysical survey data to create a magnetic susceptibility-geophysical data set;
mapping the target zone based on the magnetic susceptibility-geophysical data set to improve exploration for geologic hydrogen; and
generating, based on the identification of the magnetic anomaly, a drilling plan for accessing the target zone of the geological hydrogen source rock.

2. The method of claim 1, wherein the step of collecting magnetic susceptibility data comprises measuring the magnetic susceptibility at the plurality of subsurface locations using a vibrating sample magnetometer, a superconducting quantum interference device magnetometer, a magnetic property measurement system, an AC susceptometer, a Kappabridge, a Faraday balance, a Gouy balance, an alternating gradient magnetometer, or a wireline magnetic susceptibility tool.

3. The method of claim 1, wherein the step of collecting further comprises measuring the magnetic susceptibility along a depth of a well or borehole.

4. The method of claim 1, wherein the step of collecting the magnetic susceptibility data comprises:
obtaining a plurality of geological samples of geological hydrogen source rock within the region, each geologic sample taken at from one subsurface location of the plurality of subsurface locations; and
measuring the magnetic susceptibility of each geological sample using a laboratory or field-based instrument on the geological sample.

5. The method of claim 1, wherein the geophysical survey data comprises regional magnetic survey data collected by a ground-based magnetic method, a sea-based magnetic method, or an airborne magnetic method.

6. The method of claim 5, wherein the magnetic survey uses one or more of a scalar magnetometer, a vector magnetometer, or a magnetic gradiometer.

7. The method of claim 1, further comprising generating, based on the magnetic susceptibility-geophysical data set, a map of the region using a statistical model.

8. The method of claim 7, wherein the statistical model comprises a Werner deconvolution model.

9. The method of claim 8, wherein the Werner deconvolution modeling is one of inverse modeling or forward modeling.

10. The method of claim 1, further comprising identifying lithologic intervals using the magnetic susceptibility.

11. The method of claim 1, further comprising determining a mineral composition of each geological sample.

12. The method of claim 1, further comprising differentiating rock types.

13. The method of claim 1, further comprising measuring a wireline density at a location from which the geological sample was taken.

14. The method of claim 1, wherein the geophysical survey data comprises one or more of mineralogical data, magnetic field data, magnetic field gradient data, gravitational data, gravitational gradient data, spatially resolved well data, wireline data, wireline instrumentation data, wireline density data, acoustic impedance data, seismic data, or electromagnetic data.

15. The method of claim 14, wherein the geophysical survey data comprises airborne gravity or gravity gradiometers, airborne gravity and magnetics, ground-based gravity and magnetics, or sea-based gravity and magnetics, or electromagnetic surveys integrated with seismic reflection data.

16. The method of claim 15, further comprising identifying lithological zones corresponding to a target zone of potential hydrogen production or hydrogen accumulation.

17. The method of claim 1, wherein the target zone is capable of producing a hydrogen gas product exhibiting a carbon intensity score less than 3.0 kg CO2eq/kg H2.

18. The method of claim 17, wherein the hydrogen gas product exhibits a carbon intensity score of less than 1.5 kg CO2eq/kg H2.

19. The method of claim 18, wherein the hydrogen gas product exhibits a carbon intensity score of less than 0.45 kg CO2eq/kg H2.

20. The method of claim 1, further comprising:
training, using the magnetic susceptibility data from the plurality of subsurface locations and the geophysical survey data, a statistical model to generate a geologic map of the region, wherein the geologic map of the region includes the target zone of the geological hydrogen source rock, wherein the geologic map of the region includes above-ground characteristics or subsurface characteristics; and
identifying, using the statistical model, a further region including a target zone of the geological hydrogen source rock having a potential for hydrogen production or a potential for hydrogen accumulation, wherein the further region exhibits the above-ground characteristics or the subsurface characteristics corresponding to the above-ground characteristics or subsurface characteristics of the geologic map of the region.

21. The method of claim 1, wherein the geological hydrogen source rock of the target zone has a magnetic susceptibility greater than 0.0001.

22. The method of claim 21, wherein the geological hydrogen source rock of the target zone has a magnetic susceptibility greater than 0.004.
